(19) United States
(12) Patent Application Publication
(10) Pub. No.: US 2005/0154536 A1
(43) Pub. Date: Jul. 14, 2005

(54) Two-part drug discovery system

(75) Inventors: Carson C. Chow, Bethesda, MD (US); Yoram Vodovotz, Sewickley, PA (US); Gilles Clermont, Fombell, PA (US); Mitchell P. Fink, Pittsburgh, PA (US); John Bartels, Pittsburgh, PA (US); Steven Chang, Pittsburgh, PA (US)

Correspondence Address: THE WEBB LAW FIRM, P.C., 700 KOPPERS BUILDING, 436 SEVENTH AVENUE, PITTSBURGH, PA, 15219, US

(73) Assignee: The University of Pittsburgh - of the Commonwealth System of Higher Education, Pittsburgh, PA (US)

(21) Appl. No.: 10/925,862

(22) Filed: Aug. 25, 2004

Related U.S. Application Data
(63) Continuation-in-part of application No. 10/233,166, filed on Aug. 30, 2002.
(60) Provisional application No. 60/498,178, filed on Aug. 26, 2003.
(60) Provisional application No. 60/318,772, filed on Sep. 12, 2001.
(60) Provisional application No. 60/316,181, filed on Aug. 30, 2001.

(51) Int. Cl. G06F 19/00; G01N 33/48; G01N 33/50

(52) U.S. Cl. 702/19

(57) ABSTRACT

A mathematical prognostic in which changes in a number of physiologically significant factors are measured and interpolated to determine a “damage fluction” incident to bacterial infection or other serious inflammation, followed by either or both of in vitro or in vivo investigations of a particular active agent (drug) and adjustment of the model so as better to evaluate the particular active agent. By measuring a large number of physiologically significant factors including, but not limited to, Interleukin 6 (IL-6), Interleukin 10 (IL-10), Nitric Oxide (NO), and others, it is possible to predict life versus death by the damage function, dD/dt. To evaluate one or more drug candidates against inflammation, the mathematical model is applied first, followed by in vivo and/or in vitro investigations, and the in vivo and/or in vitro investigations are in turn used to adjust or to enhance, if applicable, the mathematical model as it is applied to the particular drug candidate.

### CROSS REFERENCE TO RELATED APPLICATIONS

This application claims the benefit of U.S. Provisional Application Ser. No. 60/316,181, filed Aug. 30, 2001, and U.S. Provisional Application Ser. No. 60/318,772, filed Sep. 12, 2001, which are incorporated by reference in their entirety, by virtue of this application's being a continuation-in-part of U.S. application Ser. No. 10/233,166 filed Aug. 30, 2002. This application also claims the benefit of U.S. Provisional Application Ser. No. 60/498,178, filed Aug. 26, 2003, which is likewise incorporated herein by reference.

### STATEMENT REGARDING FEDERALLY SPONSORED RESEARCH

This invention was made in part with Government support under NIGMS Grant Nos. RO1-GM-67240 and P50-GM-53789. The Government may have certain rights in this invention.

### BACKGROUND OF THE INVENTION

1. Field of the Invention

The present invention relates to a dynamical system of differential equations involving key components and interactions of the acute inflammatory response, allowing for interpretation of the inflammatory response in order to predict appropriate patient therapy, applicable drugs for patient therapy, and the proper timing for drug delivery. More particularly, the invention also pertains to a two-part drug discovery system that incorporates, sequentially, the use of the differential equations and their applications to project inflammatory disease outcomes and a particular approach incorporating cell culture and animal studies in order to verify and expand on the mathematical model of inflammatory disease thus deployed. This invention is designed to improve the process of rational drug design by an iterative strategy that involves the use of a mathematical model of acute inflammation, coupled with selected in vitro and in vivo experiments.

2. Description of Related Art

Recent advances in the understanding of the systemic inflammatory response syndrome (SIRS), which is also known as sepsis, and multi-system organ dysfunction syndrome (MODS) have resulted through identification of individual components of the complicated signaling pathways and structures of the immune system by genetic and biochemical means. Systemic inflammatory response syndrome (SIRS) results from a number of symptoms manifested by patients that have sustained major systematic insults, such as trauma and infection. SIRS is outwardly characterized by a combination of fever, tachycardia, tachypnea, and hypotension. MODS may originate from a poorly controlled inflammatory response resulting in cellular dysfunction, which results in macroscopic organ system dysfunction. However, the sequence of events leading to a state of persistent inflammatory response remains unclear even though much is known about the inflammatory response.

The inflammatory response results from the dynamic interaction of numerous components of the immune system in an attempt to restore homeostasis. The homeostatic balance can be upset primarily by direct tissue injury, such as mechanical trauma, pancreatitis, tissue hypoxia, and antigenic challenge resulting from infection. In restoring homeostasis caused by infection, the immune response involves several components, which include bacteria, bacterial pro-inflammatory substances, effector cells (macrophages and neutrophils), and effector cell-derived pro- and anti-inflammatory substances. Each component plays a unique role in the immune response to infection.

Bacteria and other agents stimulate the inflammatory response, directly or indirectly, by secreting certain products, or by the bacteria's own destruction and subsequent liberation of pro-inflammatory substances such as endotoxins. The arrival of bacteria is detected by a limited number of receptors on effector cells, which are the primary mediators of the inflammatory response.

Effector cells include neutrophils, monocytes, fixed tissue macrophages, lymphocytes, and vascular endothelial cells. Effector cell products play an integral role in the immune response and include reactive oxygen, nitrogen metabolites, eicosanoids, cytokines, and chemokines acting in an autocrine, paracrine, or endocrine fashion. Specifically, macrophages are multifunctional effector cells that play a central role in the acute inflammatory response. Macrophages are present a priori as sentinels in virtually all body tissues and, therefore, are chronologically the first responders to body insult or invasion. As a cellular population, macrophages are known to remain in a persistent state of activation while multi-system organ failure is developing. In the state of activation, macrophages secrete high levels of products such as cytokines, free radicals, and degradative enzymes. In addition to macrophages, neutrophils have an important role in the inflammatory response. Neutrophils are the most common leukocyte and are attracted to sites of injury and infection. Neutrophils are activated by bacterial products, such as peptides containing formylated methionine residues.

Bacteria and tissue injury also activate the complement pathway, causing the liberation of powerful neutrophil chemo-attractants such as C3a and C5a. These activated complement pathway molecules, in turn, activate neutrophils causing increased adhesiveness, tissue migration, degranulation, and phagocytosis of bacteria. Nafve neutrophils reach compromised tissue by detecting specific surface signals on vascular endothelium and navigate to their complement and subsequent activation of neutrophils. The activated complement pathway molecules also activate macrophages.

Cytokines are protein hormones that have a signaling role, primarily among immune cells and between immune cells and either endothelial or epithelial cells. Cytokines exert a vast array of effects on growth, development, immunity, and diseases that are regulated in complex ways at the transcriptional, post-transcriptional, translational, and post-translational levels. A variety of cellular products that are essential to a successful immune response to the stress are expressed as a result of the direct action of cytokines. The systemic action of cytokines as part of an activated immune system internally drives the systemic inflammatory response syndrome.

Often overlapping in their spectra of action, cytokine activities include interaction with one another, and regulation of each other's expression and activity. Pro-inflammatory cytokines, such as Tumor Necrosis Factor (TNF)-α, Interleukin (IL)-1, and Interleukin (IL)-6, are involved in various stages of the inflammatory response to microbial pathogens and their secreted products. Pro-inflammatory cytokines are made by and regulate the activity of macrophages and neutrophils. Anti-inflammatory cytokines are the counterbalancing force to pro-inflammatory cytokines and include Interleukin (IL)-10 and Transforming Growth Factor (TGF)-β1. Anti-inflammatory cytokines serve to dampen the inflammatory response and hence the return to homeostasis. However, anti-inflammatory cytokines can lead to suppression of the immune system when dysregulated.

Free radicals and degradative enzymes comprise another component of the immune response and are produced by macrophages and neutrophils. Free radicals such as superoxide, hydroxyl radical, and hydrogen peroxide, which are known collectively as reactive oxygen species, are directly toxic to pathogens and host cells. These molecules also serve a signaling role by inducing the production of pro-inflammatory cytokines. The free radical nitric oxide and the products derived from its reaction with numerous molecules, including reactive oxygen species, are known collectively as reactive nitrogen species. (The blood ionic form of reactive nitrogen species is Nitrate [NO₃⁻] and Nitrite [NO₂⁻].) These molecules can be cytotoxic or cytostatic to pathogens, and may help protect host cells from damage. However, the elevated levels of nitric oxide produced systemically upon infection can have adverse hemodynamic effects. In addition, degradative enzymes found in the granules of both neutrophils and macrophages serve to break down engulfed bacteria, and indirectly serve a signaling role by causing the release of bacterial products that, in turn, are pro-inflammatory.

Advances in understanding of the mediators of the inflammatory response have led to mechanistic rationales for the development of targeted treatments in sepsis and other diseases characterized by uncontrolled inflammation. Currently, several molecular targets are being investigated for the treatment of destructive inflammation. The therapeutic agents under investigation are anti-cytokine antibodies, soluble cytokine receptors, cyclooxygenase inhibitors, neutrophil-endothelial adhesion blockers, nitric oxide donor or scavenger molecules, and modulators of the coagulation cascade (coagulation is stimulated following both infection and trauma, and stimulates many of the inflammatory pathways described above). Despite promising results in animal and human trials, large-scale trials of therapies targeted at inhibiting or scavenging various inflammatory mediators at the global inflammatory response have generally failed to improve survival (except for a single drug, recombinant human activated protein C, known as drotrecogin alfa [activated]). Although many reasons such as the wrong rationale, questionable drug activity, faulty patient selection, and insensitive end-points, may explain the failure of the trials, the most likely explanation is that acute inflammation represents the highly integrated response of a complex adaptive immune system. Targeting one sub-mechanism of the inflammatory response will result, at best, in a modest modulation of the integrated inflammatory response.

The complexity of the molecular and genetic pathways involved in the acute response to injury has resulted in confining experimentation to the isolated aspects of the innate immune response, and intimidation about gaining an integrated description of the acute inflammatory response. Although there have been advances in understanding the complex molecular physiology of the acute inflammatory response, the reasons underlying the immune system pathways and the association between molecular events and organ dysfunction remain elusive. There has been no published attempt to model the acute inflammatory response quantitatively, presumably because of the perceived untenable complexity of the physiological response. Mathematical models that include several possible mechanisms relating inflammatory effectors and end organ damage could provide a means to correlate time-dependent patterns of effectors with outcome.

The inflammatory response to bacterial infection can be modeled by using a system of differential equations that expresses the time variations of individual components simultaneously. Such a dynamic systems approach can provide an intuitive means to translate mechanistic concepts into a mathematical framework, be analyzed using a large body of existing techniques, be numerically simulated easily and inexpensively on a desktop computer, provide both qualitative and quantitative predictions, and allow for the systematic incorporation of higher levels of complexity. Therefore, there is a present need for a simplified system of mathematical equations that involves key components and interactions of the acute inflammatory response to predict which patients are to be treated, the drugs to use to treat those patients, and the proper timing for delivery of the drugs.

### SUMMARY OF THE INVENTION

In order to meet this need, the present invention is a mathematical prognostic and model in which changes in a number of physiologically significant factors are measured and interpolated to determine a “damage function” incident to bacterial infection or other serious inflammation. By measuring a large number of physiologically significant factors including, but not limited, to Interleukin 6 (IL-6), Interleukin 10 (IL-10), Nitric Oxide (NO), and others, it is possible to predict life versus death by the damage function, dD/dt (i.e., the change in damage over time), which measures and interpolates differential data for a plurality of factors. Certain ratios of these physiologically significant factors, measured at given points in time, are representative of the damage function without embodying the damage function in its entirety, but the ratios are useful nonetheless. For example, in mammals an IL-6/NO ratio <8 at 12 hours post infection is highly predictive (60%) of mortality; also in mammals an IL-6/NO ratio <4 at 24 hours post infection is highly predictive (52%) of mortality; and an IL-6/IL-10 ratio in mammals of <7.5 at 24 hours post infection is highly predictive (68%) of mortality. This model has demonstrated its utility in simulating acute inflammation induced in mice by endotoxin, surgical trauma, and surgery/hemorrhage. Its predictive ability was tested in Trauma (sham surgery/surgical instrumentation) followed or not by Hemorrhagic Shock+LPS given at 0.5, 3, or 27 hrs after the beginning of surgical instrumentation. Either by determination of the damage function in entirety, or by observation of the IL-6/NO and/or IL-6/IL-10 levels at appointed times, prognosis of patient outcome is possible which prognosis, in turn, suggests appropriate intervention. As a model for active agent analysis, the mathematical model and the damage function, in particular, may be used to create simulated clinical trials. In these trials, variability in the patient population can be created by generating random variations in production of pro- and anti-inflammatory cytokines as well as NO in response to infection or trauma (with said variations occurring over known ranges in humans), these “virtual patients” may be subjected to simulated infection or injury at various random levels (with said variations occurring over known ranges in humans), as well as simulated standard medical interventions (e.g. antibiotics) commensurate with the degree of infection/trauma. Because the mathematical model can simulate both complex scenarios similar to real sepsis as well as simpler paradigms of inflammation (such as infusion of a defined dose of a bacteria-derived immunostimulant in either animals or humans), real patient data from bacterial infection situations is analyzed and analogized to animal model studies of active agents in order to amplify the significance of the animal model results. Also provided is a two-part drug discovery system that deploys the above mathematical model and augments it with animal studies in which controlled inflammatory response in an animal, incident to treatment with one or more active agents, is used both to confirm and to expand the mathematical model described above.

### BRIEF DESCRIPTION OF THE DRAWINGS

FIG. 1 shows several graphs illustrating the time-dependent behavior of the system;

FIG. 2 shows several graphs illustrating neutrophil with a reduced oxidative burst capacity (i.e., deficiency in the enzyme required to produce superoxide) as being quite deficient in producing pro-inflammatory cytokines;

FIG. 3 shows graphs illustrating a high baseline concentration of anti-inflammatory mediators leading to reduced expression of pro-inflammatory substances and effectors;

FIG. 4a shows several graphs illustrating the effect of pathogen inoculum size on pathogen multiplication;

FIG. 4b shows several graphs illustrating pathogen growth effect;

FIG. 4c shows a graph illustrating bifurcation, which is the irreversible impact on blood pressure caused by pathogen growth rate;

FIG. 5 shows several graphs illustrating the possibility of therapeutic intervention simulating the administration of an antibiotic through the convergence of several parameters of the system in a complicated, but suggestive, manner for a quantitative evaluation of the impact of therapeutic strategies;

FIG. 6 shows a graph illustrating the use of the system to predict the effects of administration of a substance that “soaks” the nominal endotoxin;

FIG. 7 shows the experimental data (filled circles) from C57BI/6 mice given a sub-lethal (3 mg/kg) dose of LPS;

FIG. 8 shows the results for a dose of 6 mg/kg LPS. Circulating levels of TNF and IL-10 increase rapidly and decay quickly, whereas IL-6 levels peak at approximately 2-3 h and decay more slowly;

FIG. 9 shows additional data which account for the saturation of IL-6 for LPS levels byond 6 mg/kg (see also FIG. 8;

FIG. 10 shows that surgical trauma alone resulted in elevated circulating levels of certain measured cytokines; and

FIG. 11 shows certain effects of combined surgery and hemorrhage.

### DESCRIPTION OF THE PREFERRED EMBODIMENTS

As described above, the present invention is a mathematical model in which changes in a number of physiologically significant factors are measured and interpolated to determine a “damage function” incident to bacterial infection or other serious inflammation. By measuring a large number of physiologically significant factors including, but not limited to, Interleukin 6 (IL-6), Interleukin 10 (IL-10), Nitric Oxide (NO), and others, it is possible to predict life versus death by the damage function, dD/dt, which measures and interpolates differential data for a plurality of factors. Certain ratios of these physiologically significant factors, measured at given points in time, are representative of the damage function without embodying the damage function in its entirety, but the ratios are useful nonetheless. For example, in mammals an IL-6/NO ratio <8 at 12 hours post infection is highly predictive (60%) of mortality; also in mammals an IL-6/NO ratio <4 at 24 hours post infection is highly predictive (52%) of mortality; and an IL-6/IL-10 ratio in mammals of <7.5 at 24 hours post infection is highly predictive (68%) of mortality. Either by determination of the damage function in entirety, or by observation of the IL-6/NO and/or IL-6/IL-10 levels at appointed times, prognosis of patient outcome is possible which prognosis, in turn, suggests appropriate intervention. As a model for active agent analysis, the mathematical model and the damage function, in particular, may be used to create simulated clinical trials. In these trials, variability in the patient population can be created by generating random variations in production of pro- and anti-inflammatory cytokines as well as NO in response to infection or trauma (with said variations occurring over known ranges in humans), these “virtual patients“may be subjected to simulated infection or injury at various random levels, as well as simulated standard medical interventions (e.g. antibiotics) commensurate with the degree of infection/trauma. Because the mathematical model can simulate both complex scenarios similar to real sepsis as well as simpler paradigms of inflammation (such as infusion of a defined dose of a bacteria-derived immunostimulant in either animals or humans), real patient data from bacterial infection situations is analyzed and analogized to animal model studies of active agents in order to amplify the significance of the animal model results. Also provided is a two-part drug discovery system that deploys the above mathematical model and augments it with cell culture and animal studies in which controlled inflammatory response in an animal, incident to treatment with one or more active agents, is used both to confirm and to expand the mathematical model described above.

Stated another way, the present invention is a simplified system of differential equations that incorporates key components and interactions of the acute inflammatory response to predict which patients are to be treated, the drugs to use to treat those patients, and the proper timing for delivery of the drugs. The system is capable of making specific clinical predictions for treating the early response to external biological challenges while taking into account several of the main effector mechanisms currently known in a manner that will minimize D (i.e., minimize global tissue damage/dysfunction). The system can be used to predict the outcome of common clinical interventions performed as part of the management of patients with SIRS as well as reanalyzing the data from previously published studies on sepsis. The system includes variables that recognize the possibility of clinical interventions, such as antibiotics or other molecular therapies. In addition, the system includes variables that recognize the generation of antibiotic resistance, which is a major clinical problem in the management of SIRS.

A mathematical model has been developed, that that takes into account well-vetted cellular and molecular mechanisms, and that has been calibrated in mouse endotoxemia, surgery, and surgery/hemorrhage. To repeat, included in this mathematical model is a parameter called “damage/dysfunction” (D, or more accurately dD/dt), which is modulated by various elements of inflammation, but that importantly is itself a driver of inflammation. Indeed, though this parameter currently does not have a direct molecular correlate, the mathematical model has been calibrated with the effects of this parameter accounted for. This has resulted in a very good fit of the model to the experimental scenarios described above, and the model has been able to predict the course of inflammation in mice subjected to combination insults (“multiple hits”).

Systems software can be designed to implement the system to assist clinicians in the management of patients with SIRS. The designed software could implement a standard program capable of being run on a computer, such as a web-based program, in the form of a bedside workstation device, or as a wireless handheld device to be used by the treatment team. The devices could interface with the hospital's patient database to provide real-time diagnostic data for processing by the system to suggest courses of treatment. The system could also be applied in distance consulting, wherein data could be collected from a patient from a remote location and inputted into the software implementing the system, so that a consulting physician could suggest therapies for a specific patient. When the mathematical model is confirmed and possibly augmented by sequentially using an animal model as well, as discussed above, the strategy to be used for rational design of anti-inflammatory drugs, targeting various aspects of the inflammatory cascade described by the mathematical model of acute inflammation, is to minimize D. This is accomplished by first using a computerized algorithm to search the parameter space of the mathematical model of acute inflammation, in order to determine what changes to the parameters characteristic of the inflamed state (in which D is high) will result in reducing D to levels characteristic of health. The iterative strategy would include verification of the effects of the drug on the various parameters both in vitro and in vivo, with verification of the reduction of D in vivo.

When the mathematical model is used by itself, an automated patient management system would act on diagnostic data input to deliver the appropriate treatment to a septic patient. This system would have self-correcting capabilities, adjusting the timing and dosage of interventions as the patient's condition changes. Such a system could act to stabilize a patient prior to standard hospital care. Such a system might be envisioned to be of use in military applications and remote locations as well as to paramedic personnel in civilian settings. In addition, the automated patient system could be used for offering consulting services.

The current management of a patient suffering from acute injury or infection is largely resuscitative and supportive of organ function, such as mechanical ventilation, vasopressor medications, dialysis, etc. Active interventions consist of antibiotic administration and surgery, which are performed based on limited data and understanding and are often administered without sufficient understanding of the dynamic processes that are occurring in a patient.

The system in the present invention, if translated to any of the possible devices described, would enable clinicians to intervene much more effectively in order to treat a patient with SIRS. Currently, clinical trials testing candidate drugs for treatment of the underlying inflammatory response caused by SIRS have failed to prove effective. The trials have failed to take into consideration the dynamic nature of SIRS in an individual patient, and have not been set up to address fluctuations the parameters accounted for in the present invention. Clinical trials would benefit from a rational prediction of the type and timing of interventions to perform in an individual patient. Therefore, the present invention would improve the state-of-the-art in design and implementation of clinical trials by allowing individualization of treatment. At a minimum, the present invention would rule out types of interventions that are unlikely to succeed, and identify viable therapies that would maximize efficacy of treatment.

The system includes time variations of individual components simultaneously. This approach provides an intuitive means to translate mechanistic concepts of the inflammatory response into a mathematical framework. The inflammatory response can be analyzed using a large body of existing techniques that can be numerically simulated easily and inexpensively on a desktop computer. The inflammatory response provides qualitative and quantitative predictions and allows for the systematic incorporation of higher levels of complexity. The system also gives consideration to the characteristics of pathogens and the host because a considerable amount of information is available on the kinetics of individual pathogens and antibiotic responsiveness. These variables are contained in the equations of the system that can be optimized for each individual during an initial observation phase.

Generally, the system is comprised of multiple differential equations, which describe the interaction between initiator, effector, and target components of the early inflammatory response. In combination, the differential equations constitute an algorithm to predict a patient's local and systemic response to a localized infection. The variables in the equations are described in Table 1. The interaction between the different components of the dynamical system is based on a principal of mass-action kinetics.

In the first embodiment, the system is comprised of the following 11 differential equations:

$$\begin{matrix}\frac{ⅆ p}{ⅆ t} = {k}_{p1} p ( 1 - {k}_{p2} {p}^{2} ) - ( {k}_{pm} {f}_{2} ( {m}_{a} , {T}_{ma} ) + & ( 1 ) \\ ( {k}_{pne} f ( {n}_{e} , {T}_{ne} ) ) p - {k}_{p A} Ap + P ( t ) & \\ \frac{ⅅ {p}_{c}}{ⅆ t} = {k}_{pc1} p ( {k}_{pm} f ( {m}_{a} , {T}_{a} ) + {k}_{pne} f ( {n}_{e} , {T}_{ne} ) ) + & ( 2 ) \\ {k}_{pc2} p - {k}_{pc3} {p}_{c} + C ( t ) & \\ \frac{ⅆ {p}_{c}}{ⅆ t} = {k}_{pe1} p ( {k}_{pm} f ( {m}_{a} , {T}_{a} ) + {k}_{pne} f ( {n}_{e} , {T}_{ne} ) ) + {k}_{pe2} p - {k}_{pe3} {p}_{e} & ( 3 ) \\ \frac{ⅆ {m}_{a}}{ⅆ t} = {m}_{a} ( 1 - {k}_{ma3} {m}_{a}^{2} ) ( {k}_{m1c} f ( p + {p}_{c} , {T}_{p} ) + {k}_{m1e} f ( {p}_{e} , {T}_{pe} ) + & ( 4 ) \\ {k}_{mnp} f ( {n}_{p} , {T}_{np} ) ) - {k}_{ma2} {m}_{a} + {C}_{m} & \\ \frac{ⅆ n}{ⅆ t} = n ( 1 - {k}_{n4} {n}^{2} ) ( {k}_{n1c} f ( p + {p}_{c} , {T}_{p} ) + {k}_{n1e} f ( {p}_{e} , {T}_{pe} ) + & ( 5 ) \\ n ( - {k}_{n2} {n}_{e} - {k}_{n3} ) + {C}_{n} & \\ \frac{ⅆ {n}_{e}}{ⅆ t} = ( 1 - f ( {n}_{a} , {T}_{na} ) / {T}_{na} ) ( {k}_{ne1} n + {k}_{ne2} {m}_{a} ) f ( {n}_{p} , {T}_{p} ) - {k}_{ne3} {n}_{e} & ( 6 ) \\ \frac{ⅆ {n}_{p}}{ⅆ t} = ( 1 - f ( {n}_{a} , {T}_{na} ) / {T}_{na} ) ( {k}_{ne1} n + {k}_{np2} {m}_{a} ) f ( {n}_{p} , {T}_{p} ) - {k}_{np3} {n}_{p} & ( 7 ) \\ \frac{ⅆ {n}_{a}}{ⅆ t} = {n}_{ar} - {k}_{na4} {n}_{a} & ( 8 ) \\ \frac{ⅆ {n}_{ar}}{ⅆ t} = - {k}_{na1} {n}_{ar} + {k}_{na2} f ( n , {T}_{n} ) - {k}_{na3} f ( {m}_{a} , {T}_{ma} ) & ( 9 ) \\ \frac{ⅆ B}{ⅆ t} = - ( {k}_{bp1} {p}_{e} + {k}_{bp2} {n}_{p} ) \dot{B} + {B}_{0} - B & ( 10 ) \\ \frac{ⅆ A}{ⅆ t} = - a {k}_{p1} A + S ( t ) & ( 11 )\end{matrix}$$

Equation 1 describes the population behavior of pathogens. A bacterial pathogen P is externally introduced within the time course C(t) and multiplies exponentially. The system conceptually includes the property of macrophages mₐ as well as neutrophils n and reactive oxygen and nitrogen species nₑ, which is a killing substance released by both macrophages mₐ and neutrophils n.

Equation 2 describes the different mechanisms by which pathogens cause inflammation. The pathogens promote inflammation through a complement-like substance p_{c} and an endotoxin-like substance pₑ. Pathogens coated with a complement-like substance p_{c} attract the effector cells and stimulate the activation of the stimulator cells.

Equation 3 describes the sequence of interactions surrounding the liberation and localized spread of endotoxin pₑ induced by bacterial pathogens. Although endotoxins pₑ accompanies live pathogens, destruction of pathogens by macrophages mₐ, neutrophils n, and eventually antibiotic agents is related to temporary increase in the liberation of endotoxins pₑ. The intiator pₑ does not multiply, but undergoes catabolism and can efflux from the site of infection and cause inflammation in target organs. This sequence of interactions is also detailed in the relevant term of Equation 10. Although bacterial invasion is the leading paradigm of this simplified model, the inclusion of several constants in the model allows the simulation of a variety of pathogens. For example, direct tissue damage, such as trauma, would not generate intact pathogens p, but rather a complement-like effector substance pₑ according to a time dependent function C(t).

The cellular effector components included in the model are macrophages mₐ and neutrophils n. Five types of soluble effectors are also included in the model. More neutrophils n and macrophages mₐ will be activated secondarily to the presence of intact pathogens, inert soluble pathogenic components such as a complement-like substances pₑ or endotoxins pₑ, or a soluble pro-inflammatory effector substance nₚ. Activated macrophages can die at a baseline rate or be deactivated by the presence of anti-inflammatory effector substance nₐ. The macrophage dynamic is detailed in Equation 4. Neutrophils are governed by a similar dynamic, except that the rates of activation and deactivation are higher than for macrophages. In addition, it is assumed that endotoxin-like substance pₑ could activate neutrophils directly. The model allows the flexibility to separate the ability of the neutrophil to produce pro-inflammatory effector substance nₚ and the ability to release reactive oxygen and nitrogen species nₑ, because each are clearly stimulated and inhibited by different processes. This is conveyed by the use of different rates of production of these products in Equation 6 and Equation 7. The neutrophil dynamic is detailed in Equation 5. The reactive oxygen and nitrogen species nₑ, are produced by both macrophages mₐ and neutrophils n, but their ability to produce these effector molecules is saturable and modulated by the presence of soluble anti-inflammatory effector substances nₐ. This dynamic is detailed in Equation 6.

The generation of a soluble pro-inflammatory effector substance nₚ follows a similar dynamic, with different rates. The soluble anti-inflammatory substances nₐ are produced by both macrophages mₐ and neutrophils n, but their appearance is delayed with respect to pro-inflammatory effector substances. In this system, the rate of production of soluble anti-inflammatory effector substances nₐ is linked to the effector cells, not the concentration of soluble pro-inflammatory effector substances nₚ. On the other hand, the action of both soluble pro-inflammatory effector substance nₚ and soluble anti-inflammatory effector substances nₐ either shorten or prolong cell life, which reflects their respective contribution on the timing of apoptotic cell death. This dynamic is described in Equation 7, Equation 8, and Equation 9.

In the system, the model target tissue is a generic arteriole without attempting to separate smooth muscle cells and endothelium. The principle used is that the arteriole is responsible for generating the observed physiologic variable of vascular tone (as a proxy to systemic blood pressure). Vascular tone is influenced directly by effector components effluxing from the primary site of inflammation, but only once the concentration of effector agent at the primary site exceeds a predetermined threshold. It is hypothesized that soluble effectors such as endotoxins pₑ and soluble pro-inflammatory effector substances nₚ effluxed at lower concentrations than effector cells. We also assumed that soluble effectors such as endotoxins pₑ were more potent than soluble pro-inflammatory effector substances nₚ in generating a hypotensive response. This dynamic is described in Equation 10.

Finally, Equation 11 describes the dynamic of an extrinsic intervention that results in pathogen killing.

Table 1 describes the components of the acute inflammatory response as used in the first embodiment of the system.

TABLE 1
Components of the Acute Inflammatory Response included in the System

| COMPONENTS | DESCRIPTION | EXAMPLES |
|---|---|---|
| Initiator | | |
| p | Intact pathogen, can multiply | Bacteria |
| p_{c} | Inert pathogenic component that can attract and | Complement |
| | activate effector cells | |
| pₑ | Inert pathogenic component that activates effector | Endotoxin |
| | cells and be transported to distant sites | |
| Effector | | |
| mₐ | First effector cell to be activated, acts as general | Macrophage |
| | activator, produces some soluble effectors | |
| n | Second effector cell, produces soluble effectors that | Neutrophils |
| | destroys p | |
| nₑ | Soluble effector produced by n and m, kills intact | Reactive oxygen |
| | pathogens | and nitrogen |
| | | species, |
| | | degradative |
| | | enzymes |
| nₚ | Soluble “pro-inflammatory” effector | TNF-α, IL-6 |
| nₐ | Soluble “anti-inflammatory” effector | IL-10, TGF-β₁ |
| nₐᵣ | Anti-inflammatory delay variable, as these are | |
| | generally expressed later than pro-inflammatory | |
| | effectors | |
| Target | | |
| B | A physiologic observable, such as blood pressure, | Blood pressure |
| | that correlates with global outcome | |
| Intervention | | |
| A | An extrinsic modulator of the response which | Antibiotic |
| | enhances the killing of pathogen | |

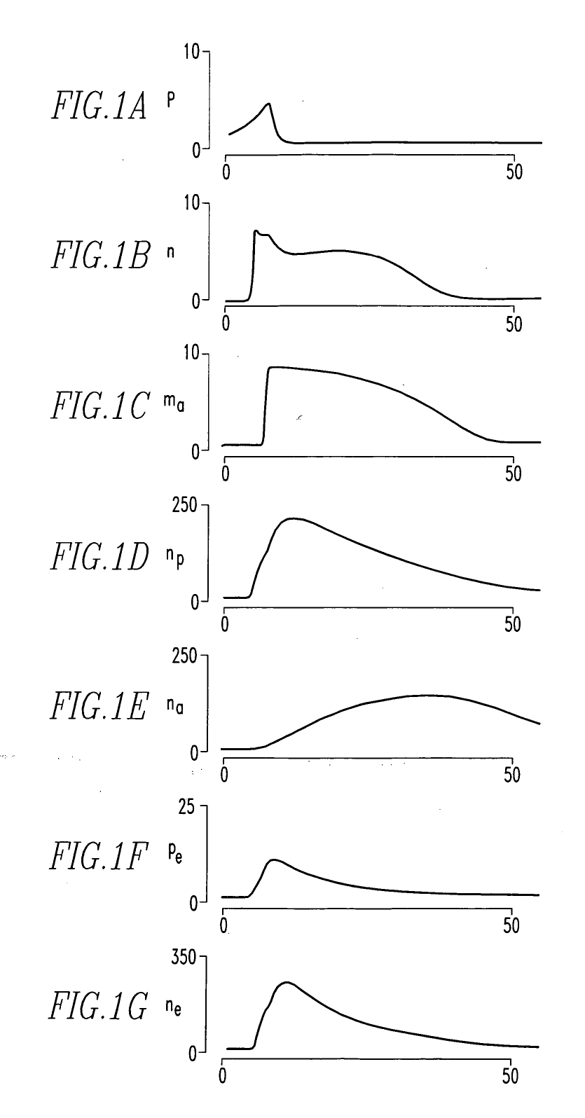

In initial experiments with the system, variables were run while considering localized processed concentration of various variables included in the model, and the effect of spill-out of effectors on blood pressure. The purpose of the initial runs was to obtain a description of events in several scenarios, reflecting common clinical situations. As shown in FIG. 1, the time-dependent behavior of the system is shown, wherein the concentrations (y-axis) and time (x-axis) are not calibrated. The usefuilness of these simulations is limited to the qualitative behavior of the system.

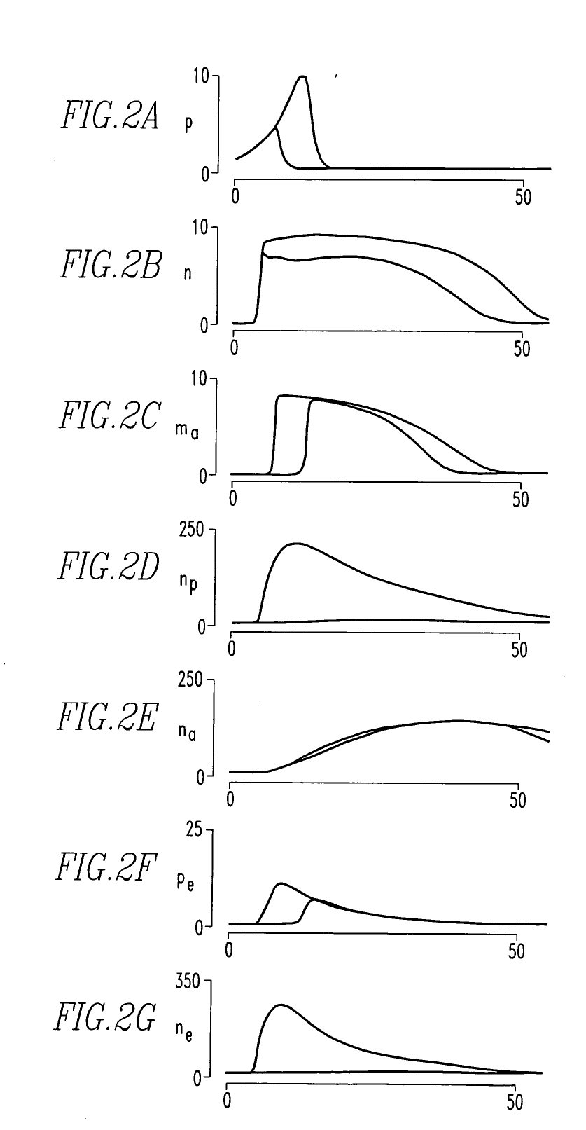

As shown in FIG. 2, a neutrophil with a reduced oxidative burst capacity (i.e., deficiency in the enzyme required to produce superoxide) is quite deficient in producing pro-inflammatory cytokines. Pathogens typically grow to a larger population, but are nevertheless cleared by the combined action of macrophages and their effectors. However, if the system simulation is allowed to run for longer time periods, pathogens reappear. This situation occurs in patients with chronic granulomatous disease.

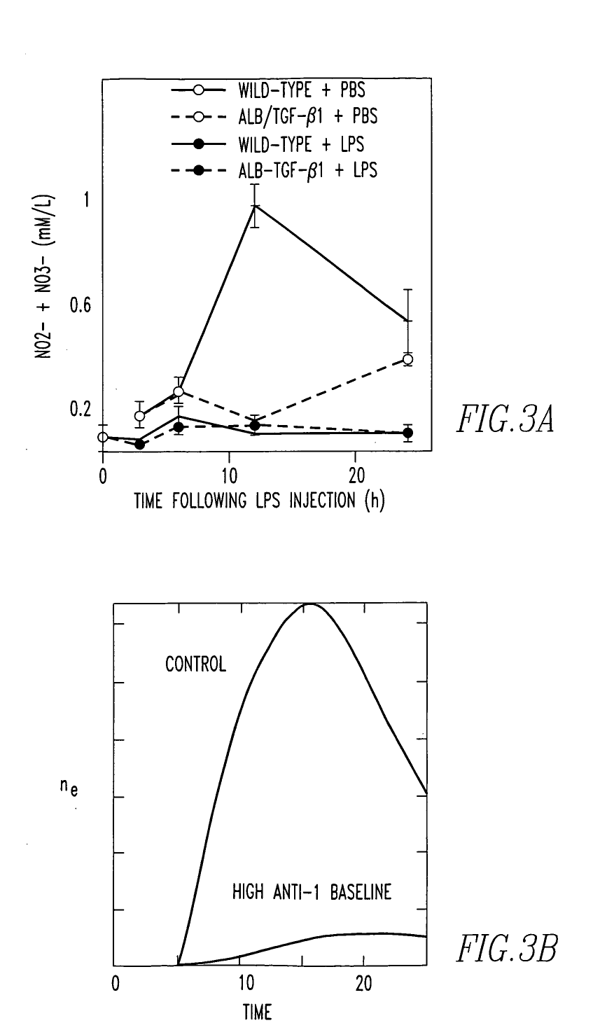

As shown in FIG. 3, a high baseline concentration of anti-inflammatory mediators leads to reduced expression of pro-inflammatory substances and effectors, such as nitric oxide. In this experiment, the over expression of TGF-β1 in mice had significantly reduced production of NO related substances (serum nitrites and nitrates) when administered lipopolysaccharide (LPS) when compared to wild-type mice or mice administered placebo (PBS). This situation occurs in some cancer patients, in patients with a natural propensity to produce TGF-β1 at a high level, or in patients previously infected with certain intracellular parasites.

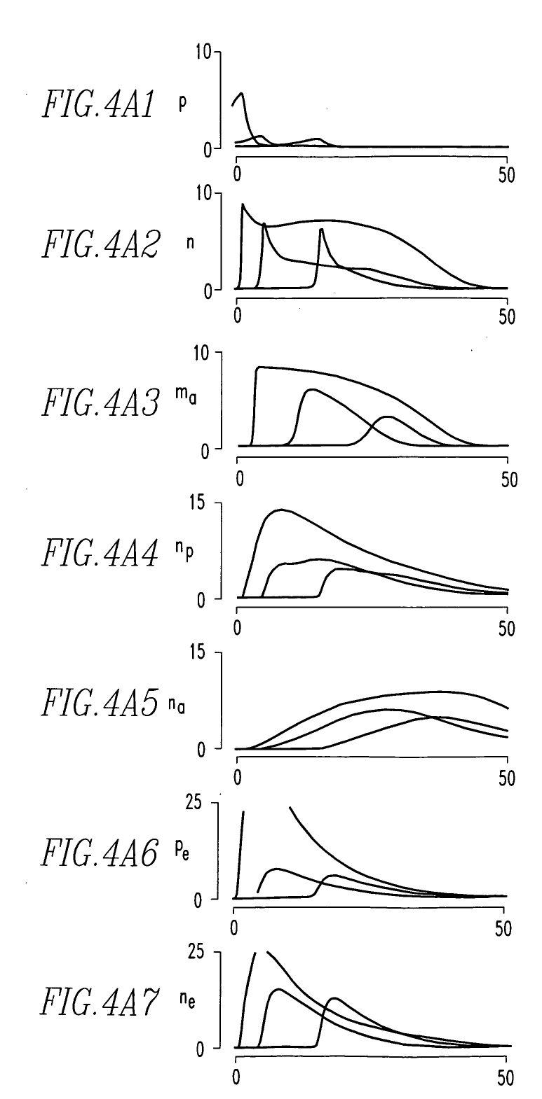

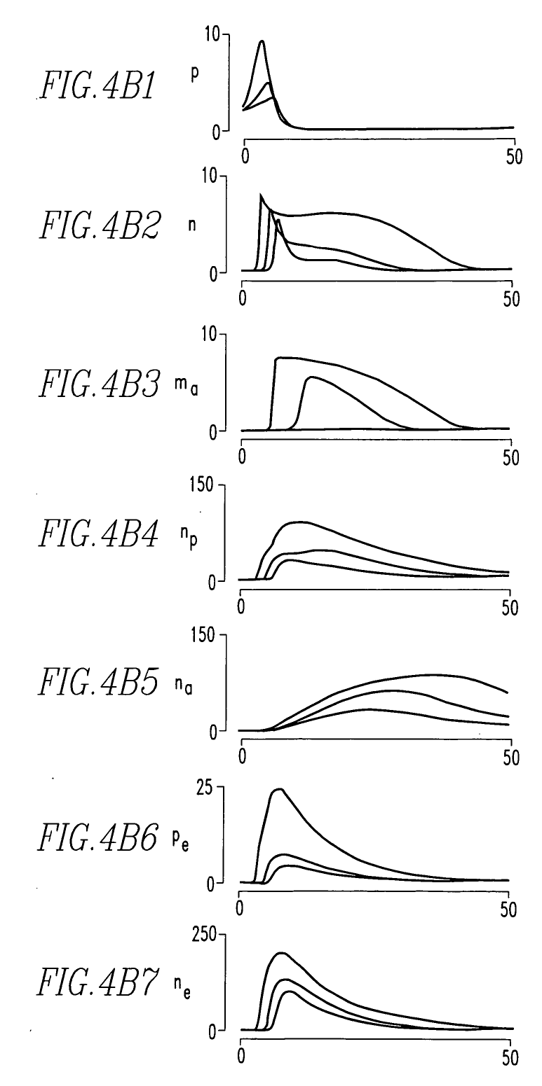

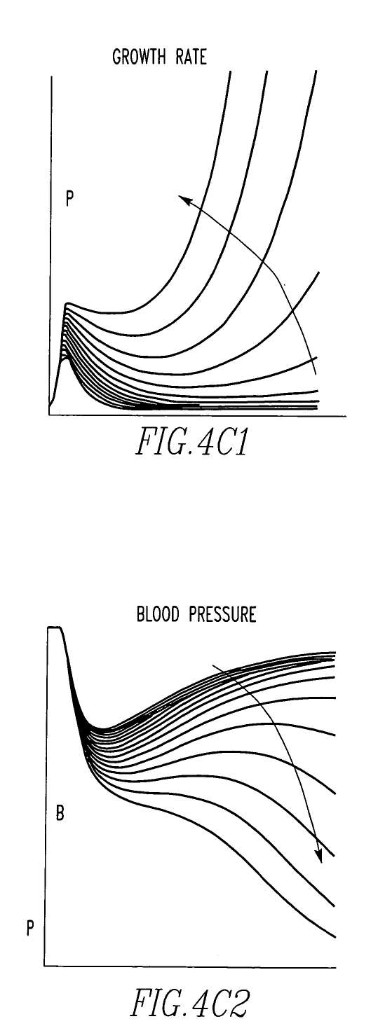

FIGS. 4a-4c show the multiplication rates of pathogens and how different sizes of pathogen inocula affect pathogen growth rates. As illustrated in FIGS. 4a-4c, the growth rate of the pathogen is clearly more important than the size of the inoculum. This information is important because the system can predict a threshold growth rate at which the immune defense mechanisms are incompetent to control the infection. The system can monitor pathogen growth and link that data with a catastrophic drop in blood pressure to show the death of a patient.

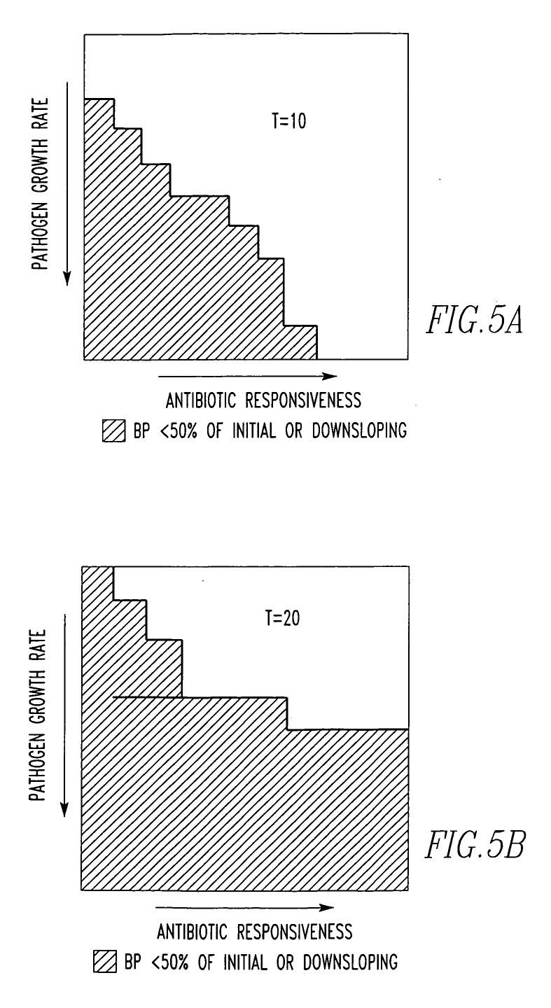

As shown in FIG. 5, a therapeutic intervention simulating the administration of an antibiotic can be used to predict the effect of a antibiotic on a patient. A substance that directly killed pathogens was introduced with a user-specific efficacy. The efficacy was decreased over time to simulate the gradual loss of efficacy of antibiotics as resistant pathogens are selected. As expected, administration of antibiotics assists in the more rapid control of an infection. An effective antibiotic will help control an infection that would otherwise be lethal. However, later intervention with an antibiotic, prior to death, will result in considerably less impact of an otherwise effective antibiotic on death. The convergence of several parameters of the system in a complicated manner can be accomplished by the system. Increased antibiotic effectiveness results in better eradication of pathogens and presumably better survival. Increased growth rate of pathogen results in worse survival. Earlier administration of antibiotic may save lives, everything else being equal. “Death“means a decrease by more than 50% of blood pressure or down-sloping of blood pressure at the end of the simulation (t=50). The simulation provides a prediction of the outcome (in blood pressure) given bacterial growth rate and antibiotic efficacy and the quantitative evaluation of the impact of therapeutic strategies in isolation or in combination.

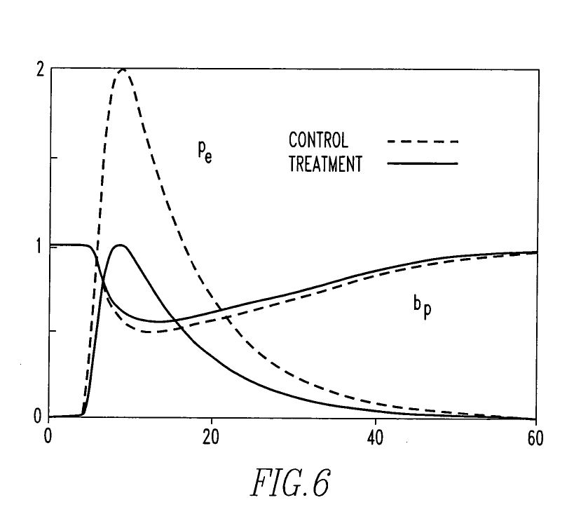

As shown in FIG. 6, the system can be used to predict the effects of administering a “soaking” substance, such as endotoxin pₑ FIG. 6 shows that the final effect on blood pressure is marginal, even though more than 50% by surface area if the endotoxin was soaked. The marginal effect on blood pressure occurs because more than one factor in the model is responsible for the decrease in blood pressure. Quantifying the relative importance of different processes to impact outcome is of paramount importance in the design of medical therapies. If endotoxin was the major factor contributing to lower the blood pressure, the results obtained from the system would show a major impact from an anti-endotoxin therapy.

In the second embodiment, the system includes a more detailed model of acute inflammation variables. The following 16 differential equations comprise the second embodiment of the system. Immediately following the second set of equations is a third embodiment comprising a set of thirteen equations listed separately.

$$\begin{matrix}\frac{ⅅ P}{ⅅ t} = {k}_{p} P ( 1 - {k}_{Ps} P ) - ( {k}_{PM} {M}_{a} + {k}_{PO2} {O}_{2} + {k}_{PNO} NO + & ( {1}^{′} ) \\ AB ( t ) ) P + {S}_{P} ( t ) & \\ \frac{ⅅ PE}{ⅅ t} = ( {k}_{P} {M}_{a} + {k}_{PO2} {O}_{2} + {k}_{PNO} NO + AB ( t ) P - {k}_{PE} PE + {S}_{PE} ( t ) & ( {2}^{′} ) \\ \frac{ⅅ {M}_{r}}{ⅅ t} = - ( {k}_{MP} p + {k}_{MPE} PE + {k}_{MD} D ) ( f ( {C}_{p} ) + f ( IL - 6 ) ) {f}_{s} ( {C}_{a} ) + & ( {3}^{′} ) \\ {k}_{Mg} f ( {M}_{a} + {C}_{p} + NO + PE ) - {k}_{M} {M}_{r} & \\ \frac{ⅅ {M}_{a}}{ⅅ t} = ( {k}_{m p} p + {k}_{pe} PE + {k}_{md} D ) ( f ( {C}_{p} ) + f ( IL - 6 ) ) {f}_{s} ( {C}_{a} ) - {k}_{ma} {M}_{a} & ( {4}^{′} ) \\ \frac{ⅅ N}{ⅅ t} = ( {k}_{NP} P + {k}_{NPE} PE + {k}_{NCP} {C}_{P} + {k}_{NIL - 6} IL - 6 + {k}_{ND} D ) N - & ( {5}^{′} ) \\ ( {k}_{NNO} NO + {k}_{NO2} O2 ) N - {k}_{N} {f}_{s} ( {C}_{p} ) N & \\ \frac{ⅅ {O}_{2}}{ⅅ t} = ( ( {k}_{O2N} N + {k}_{O2M} {M}_{a} ) ( f ( {C}_{p} ) + f ( IL - 6 ) ) + & ( {6}^{′} ) \\ {k}_{O2NP} NP ) {f}_{s} ( {C}_{a} ) - {k}_{O2} {O}_{2} & \\ \frac{ⅅ N}{\frac{O}{ⅅ t}} = ( {k}_{NON} N + {k}_{NOM} {M}_{a} ) ( f ( {C}_{p} ) + f ( IL - 6 ) ) {f}_{s} ( {C}_{a} ) - {k}_{NO} NO & ( {7}^{′} ) \\ \frac{ⅅ {C}_{p}}{ⅅ t} = ( {k}_{CpN} N + {k}_{CpM} {M}_{a} ) ( 1 + {k}_{CPn} f ( {C}_{p} ) ) {f}_{s} ( {C}_{a} ) - {k}_{Cp} {C}_{p} & ( {8}^{′} ) \\ \frac{ⅅ I {L}_{-}}{\frac{6}{ⅅ t}} = {k}_{IL - 6 M} {M}_{a} {f}_{s} ( {C}_{a} ) - {k}_{IL - 6} IL - 6 & ( {9}^{′} ) \\ \frac{ⅅ {C}_{ar}}{ⅅ t} = ( {k}_{CaN} N + {k}_{CaM} {M}_{a} ) f ( {C}_{p} + NO + {O}_{2} ) - {k}_{Car} {C}_{ar} & ( {10}^{′} ) \\ \frac{ⅅ {C}_{a}}{ⅅ t} = {C}_{ar} - {k}_{Ca} {C}_{a} + {S}_{PC} ( t ) & ( {11}^{′} ) \\ \frac{ⅅ TF}{ⅅ t} = ( {k}_{TFPE} PE + {k}_{TFCp} {C}_{p} + {k}_{TFIL - 6} IL - 6 ) {f}_{s} ( PC ) - & ( {12}^{′} ) \\ {k}_{TF} TF - ktf ( t ) TF & \\ \frac{ⅅ TH}{ⅅ t} = TF ( 1 + {k}_{THn} TH ) - {k}_{TH} TF & ( {13}^{′} ) \\ \frac{ⅆ TH}{ⅆ T} = TF ( 1 + {k}_{THn} TH ) - {k}_{TH} TF & \\ \frac{ⅅ PC}{ⅅ t} = {k}_{PCTH} TH - {k}_{PC} PC + {S}_{PC} ( t ) & ( {14}^{′} ) \\ \frac{ⅅ BP}{ⅅ t} = {k}_{BP} ( 1 - BP ) - {k}_{BPO2} {O}_{2} {f}_{s} ( NO ) + {k}_{BPCp} {C}_{p} + {k}_{BPTH} TH ) BP & ( {15}^{′} ) \\ \frac{ⅅ D}{ⅅ t} = {k}_{DBP} ( 1 - BP ) + {k}_{DCp} {C}_{p} + {k}_{DO2} {O}_{2} + {k}_{DNO} NO / ( 1 + NO ) + & ( {16}^{′} ) \\ {k}_{DEqg} ( {O}_{2} , NO ) - {k}_{D} D & \\ {M}_{R}^{′} = - [ ( {k}_{MLPS} \frac{{LPS ( t )}^{2}}{1 + {( LPS ( t ) / {x}_{MLPS} )}^{2}} + {k}_{MD} \frac{{D}^{4}}{{x}_{MD}^{4} + {D}^{4}} ) \times & {1}^{′} ’ \\ ( \frac{{TNF}^{2}}{{x}_{{MTNF}^{2}} + {TNF}^{2}} + {k}_{M6} \frac{{IL6}^{2}}{{x}_{M6}^{2} + {IL6}^{2}} ) + & \\ {k}_{MTR} TR ( t ) + {k}_{MB} {f}_{B} ( B ) ] \frac{1}{1 + {( IL10 / {x}_{M10} )}^{2}} {M}_{R} - & \\ {k}_{MR} ( {M}_{R} - {S}_{M} ) & \\ {M}_{A}^{′} = [ ( {k}_{MLPS} \frac{{LPS ( t )}^{2}}{1 + {( LPS ( t ) / {x}_{MLPS} )}^{2}} + {k}_{MD} \frac{{D}^{4}}{{x}_{MD}^{4} + {D}^{4}} ) \times & {2}^{′} ’ \\ ( \frac{{TNF}^{2}}{{x}_{{MTNF}^{2}} + {TNF}^{2}} + {k}_{M6} \frac{{IL6}^{2}}{{x}_{M6}^{2} + {IL6}^{2}} ) + {k}_{MTR} TR ( t ) + {k}_{MB} {f}_{B} ( B ) ] & \\ \frac{1}{1 + {( IL10 / {x}_{M10} )}^{2}} {M}_{R} - {k}_{MA} MA & \\ {N}_{R}^{′} = - [ ( {k}_{NLPS} \frac{LPS ( t )}{1 + LPS ( t ) / {x}_{NLPS}} + {k}_{NTNF} \frac{TNF}{1 + TNF / {x}_{NTNF}} + & {3}^{′} ’ \\ {k}_{N6} \frac{{IL6}^{2}}{1 + {( IL6 / {x}_{N6} )}^{2}} + {k}_{ND} \frac{{D}^{2}}{1 + {( D / {x}_{ND} )}^{2}} + {k}_{NB} {f}_{B} ( B ) + & \\ {k}_{NTR} TR ( t ) ) \times \frac{1}{1 + {( IL10 / {x}_{N10} )}^{2}} {N}_{R} - {k}_{NR} ( {N}_{R} - {S}_{N} ) & \\ {N}_{A}^{′} = [ ( {k}_{NLPS} \frac{LPS ( t )}{1 + {( LPS ( t ) / {x}_{NLPS} )}^{2}} + {k}_{NTNF} \frac{TNF}{1 + TNF / {x}_{NTNF}} + & {4}^{′} ’ \\ {k}_{N6} \frac{{IL6}^{2}}{1 + {( IL6 / {x}_{N6} )}^{2}} + {k}_{ND} \frac{{D}^{2}}{1 + {( D / {x}_{ND} )}^{2}} + {k}_{NB} {f}_{B} ( B ) + & \\ {k}_{NTR} TR ( t ) ) \times \frac{1}{1 + {( IL10 / {x}_{N10} )}^{2}} {N}_{R} - {k}_{N} {N}_{A} & \\ {iNOSd}^{ ′} = ( {k}_{INOSN} {N}_{A} + {k}_{INSOM} {M}_{A} + {k}_{INOSEC} & {5}^{′} ’ \\ ( \frac{{TNF}^{2}}{1 + {( TNF / {x}_{INOSTNF} )}^{2}} + {k}_{INOS6} \frac{{IL6}^{2}}{1 + {( IL6 / {x}_{INOS6} )}^{2}} ) ) \times & \\ \frac{1}{1 + {( IL10 / {x}_{INOS10} )}^{2}} \frac{1}{1 + {( NO / {x}_{iNOSNO} )}^{4}} - {k}_{INOSd} i NOSd & \\ {iNOS}^{ ′} = {k}_{iNOS} ( iNOSd - iNOS ) & {6}^{′} ’ \\ {eNOS}^{ ′} = {k}_{ENOSEC} \frac{1}{1 + TNF / {x}_{ENOSTNF}} \frac{1}{1 + LPS ( t ) / {x}_{ENOSLPS}} & {7}^{′} ’ \\ \frac{1 }{1 + {( TR ( t ) / {x}_{ENOSTR} )}^{4}} - {k}_{ENOS} eNOS & \\ {NO}_{3}^{′} = {k}_{NO3} ( NO - {NO}_{3} ) & {8}^{′} ’ \\ {TNF}^{′} = ( {k}_{TNFN} {N}_{A} + {k}_{TNFN} {M}_{A} ) \frac{1}{1 + {( ( IL10 + CA ) / {x}_{TNF10} )}^{2}} & {9}^{′} ’ \\ \frac{IL6}{1 + {( IL6 / {x}_{TNF6} )}^{3}} - {k}_{TNF} TNF & \\ {IL6}^{′} = ( {k}_{6 N} {N}_{A} + {M}_{A} ) ( {k}_{6 M} + {k}_{6 TNF} \frac{{TNF}^{2}}{{x}_{6 TNF}^{2} + {TNF}^{2}} + {k}_{6 NO} & {10}^{′} ’ \\ \frac{{NO}^{2}}{{x}_{6 NO}^{2} + {NO}^{2}} ) \frac{1}{1 + {( ( CA + IL10 ) / {x}_{610} )}^{2}} + {k}_{6} ( {S}_{6} - IL6 ) & \\ {IL12}^{′} = {k}_{12 M} {M}_{A} \frac{1}{1 + {( IL10 / {x}_{1210} )}^{2}} - {k}_{12} IL12 & {11}^{′} ’ \\ {CA}^{′} = {k}_{CATR} A ( t ) - {k}_{CA} CA & {12}^{′} ’ \\ {IL10}^{′} = ( {k}_{10 N} {N}_{A} + {M}_{A} ( 1 + {k}_{10 A} A ( t ) ) ( {k}_{10 MR} + {k}_{10 TNF} & {13}^{′} ’ \\ \frac{{TNF}^{4}}{{x}_{10 TNF}^{4} + {TNF}^{4}} + {k}_{106} \frac{{IL6}^{4}}{{x}_{106}^{4} + {IL6}^{4}} ) ( 1 - {k}_{10 R} ) & \\ \frac{1}{1 + {( IL12 / {x}_{1012} )}^{4}} + {k}_{10 R} ) - {k}_{10} ( IL10 - {S}_{10} ) & \end{matrix}$$

The equations in the second embodiment incorporate pathogen P, endotoxin Pₑ, resting and active macrophages Mᵣ and Mₐ, respectively, neutrophils N, two effector molecules NO and O₂, a short term pro-inflammatory cytokine Cₚ, a longer-term pro-inflammatory cytokine (that later induces anti-inflammatory mechanisms) JL-6, and an anti-inflammatory activity comprising multiple cytokines Cₐ. This system also includes recognition of a coagulation system represented by tissue factor TF, thrombin TH, and activated protein P_{C}. This system recognizes a blood pressure variable BP and a tissue dysfumction/damage variable D. Similar to the first embodiment, there is a source term for pathogens and endotoxins as well as an antibiotic term to eliminate pathogens. Antibiotic resistance is incorporated into the system by reducing the efficacy of pathogen elimination by antibiotics in a time-dependent way. Effective therapies, such as mechanisms for clearing pro-inflammatory cytokines, and means of enhancing the supply of anti-inflammatory cytokines and activated protein C, are included in the system. The blood pressure variable can be lowered to simulate the effects of trauma by inducing damage and hemorrhaging.

The present invention can be calibrated to capture the quantitative aspects of the object being modeled. A calibrated system is capable of estimating concentrations and the actual variations of those concentrations, or other physiologic parameters such as cell count and blood pressure, over time. The estimation of the various rates is derived from the literature, when available, or from educated guesses, and comparing the dynamic description obtained from the empirical data. The system contains approximately 50 parameters, most of which reflect the relative importance of certain processes, such as cell or effector half-lives, as well as the phenomena of biological saturation or exhaustion, where the effects of positive feedback are limited.

The system must be optimized to embrace the primary goal of the system to predict which interventions, as shown by modifications in the dynamic structure of the model, would most significantly alter a measurable outcome. For example, a decrease in blood pressure will result in death, an undesirable event in most circumstances in critically ill patients. Some parameters are static, while others can be modified within certain limits. The process of optimization involves the steps of defining the quantity to optimize, determining a selection of parameters that can be varied in the process of optimization, determining a realistic range over which any of these parameters can be varied, choosing an optimization technique, and verifying the face validity of the results of the procedure. In most circumstances of immediate concern, the initial conditions are fixed, so one is not in search of a global optimal solution, but of a local one. This is important to know, because this knowledge would dictate that interventions are futile and outcome certain, good or bad. The framework of differential equations to express non-linear dynamics is more favorable than more heuristic methods of representing the problem if optimization is a major issue. Although alternative frameworks can be created (e.g. discrete event simulation could also be used), optimizing such representations is particularly challenging.

The following disclosure explains in particular the Two-Part Drug Discovery System embraced by this patent specification.

Selection and vetting of therapeutic agents is conducted as follows, in the two-part drug discovery system. The mathematical model discussed above is used to evaluate a given active agent to describe the acute inflammatory cascade that culminates in global tissue damage/dysftnction (D). After applying the model even to the extent of optionally conducting a virtual clinical trial in silico, in which a simulated population of non-survivors is subjected to manipulations required to change their fate to that of survivors, a therapeutic agent is produced that whose features at the molecular/cellular/organismic levels are those suggested by this simulation to cause this increase in survival. Where applicable, the molecular and cellular effects of this novel therapeutic agent are tested in appropriate in vitro studies. Subsequently, an animal study is performed with the same therapeutic agent not only to verify the inflammatory mechanisms (targets) apparent from both the mathematical model and the in vitro studies, but also to investigate the possibility of further inflammatory mechanisms not predicted by the mathematical model but apparent in vivo. The efficacy of the selected agent is then subsequently tested in an appropriately modified, simulated clinical trial, in which a variable population is generated using variation in insult size/inoculum as well as variation in inflammatory products. Technically, the two-part system often becomes a three-part system in that mathematical models, in vivo experiments and in vitro investigations are used in conjunction, with either the in vivo or the in vitro investigations taking place ahead of the other but with either following initial application of the mathematical model.

The following investigation was exemplary of the above. An automated search of the parameter space of the mathematical model of inflammation suggests that a drug candidate that will reduce D sufficiently to increase survival in a lethal model of endotoxemia in mice will have the following properties: in vitro reduction of the responsiveness of macrophages to TNF as well as a reduction in the capacity of macrophages to produce TNF; in vitro elevation of the capacity of macrophages to produce active TGF-β1; in vivo reduction in serum TNF, IL-6, and NO₂⁻/NO₃⁻, and in vivo elevation of IL-10. An animal model is then used to evaluate the particular drug candidate de novo, looking not only for the predicted effects but any other effects that were not predicted by the mathematical model. The mathematical model may be modified accordingly (e.g. the values of constants adjusted based on a semi-automated fitting algorithm in order to match as well as possible the actual data obtained) and the drug candidate evaluated again by mathematical/in silico means. This drug candidate is synthesized based on these features, and then is tested for its ability to cause these effects in vitro and in vivo. With appropriate modification of the mechanism of action of this drug candidate based on these studies, a simulated clinical trial of sepsis is carried out using the mathematical model in order to predict 1) whether or not the agent would be of benefit in this more complex inflammatory scenario, 2) the dosage and timing of this agent in this patient population, and 3) the exact characteristics of the patient population in a real clinical trials (i.e., inclusion and exclusion criteria) necessary in order to achieve maximal therapeutic efficacy.

A specific candidate drug and the approach to take with it is described as follows. Reduced nicotinamide adenine dinucleotide (NAD+) is a ubiquitous cellular constituent that is used by cells in a wide variety of enzyme-catalyzed, intracellular redox reactions. Accumulating data suggest that NAD+ also functions as a signaling molecule, but the mechanisms for this effect are still unclear. In a concentration-dependent fashion, NAD+ decreased the concentrations of TNF-α and NO₂⁻/NO₃⁻ in supernatants of LPS-stimulated RAW 264.7 murine macrophage-like cells. Treating endotoxemic mice with NAD+ (132 mg/kg every 12 h) significantly improved survival (in mice challenged with a lethal dose of LPS [17 mg/kg]), and decreased circulating concentrations of the pro-inflammatory cytokines TNF-α and IL-6 and NO₂⁻/NO₃⁻, while increasing the circulating concentrations of IL-10, in mice treated with a survivable dose of LPS (3 mg/kg). Given the in vitro and in vivo actions of NAD+, and the paucity of knowledge regarding its mechanism of action, a mathematical model of acute inflammation was used to 1) obtain insights as to how this agent may exert this profile of effects, and 2) predict its actions in other inflammatory settings. This model was fit to the data in mice treated with 3 mg/kg LPS alone or in combination with 132 mg/kg NAD+ as described above. Analysis of the differences in constants obtained from the two datasets predicted that 1) the half-life of NAD+ had to be on the order of a few minutes, and 2) that a primary effect of NAD+ was the reduced production of and sensitivity to TNF-α. The mathematical model had not included intracellular signal transduction pathways explicitly. However, it was determined that incubating RAW 264.7 cells with LPS markedly increased steady-state expression of both TNF and iNOS transcripts and that NAD+ decreased the expression of both of these transcripts. Both TNF and iNOS expression in murine macrophages is partially regulated by the pro-inflammatory transcription factor, NF-κB. Thus, a mechanism of action was partially inferred using a strategy combining use of a mathematical model of inflammation along with in vitro and in vivo experiments.

A particular laboratory approach is outlined below, in support of the above assertions, in the nature of an Example.

We show that our model can account for the temporal changes in the concentrations of three selected cytokines and nitric oxide by-products in mice for disparate initial insults involving endotoxin, surgical trauma, and hemorrhage. We consider this mathematical model to be a starting point for developing an in silico “virtual patient” for which therapies can be designed and tested, and real-time outcome predictions can be made.

Materials and Methods

Mice: All animal experiments were approved by the Institutional Animal Care and Use Committee of the University of Pittsburgh. All studies were carried out in C57B1/6 mice (6-10 wk old mice; Charles River Laboratories, Charles River, Me.).

Endotoxemia protocol: Mice received either LPS (from E. coli O111 :B4, 3, 6 or 12 mg/kg intraperitoneally; Sigma Chemical Co., St. Louis, Mo.) or saline control. At various time points following this injection, the mice (4-8 separate mice per time point) were euthanized and their serum obtained for measurement of various analytes (see below). All of the mice survived this high dose of LPS until the final time point (24 h following injection of LPS).

Surgical trauma and hemorrhagic shock protocols: For surgical trauma and hemorrhagic shock treatment, mice were anesthetized and both femoral arteries were surgically prepared and cannulated. For hemorrhagic shock, the mice were then subjected to withdrawal of blood with a MAP maintained at 25 mm Hg for 2.5 h with continuous monitoring of blood pressure as described previously. The normal MAP in mice is approximately 100 mmHg. In the resuscitated hemorrhage groups, the mice were resuscitated over ten minutes with their remaining shed blood plus two times the maximal shed blood amount in lactated Ringer's solution via the arterial catheter. For trauma, only the surgical preparation was conducted. In some cases, endotoxin was administered intraperitoneally to mice undergoing hemorrhagic shock. Animals were euthanized by exsanguination at various times after surgery only or hemorrhage and resuscitation, and their serum analyzed as described below.

Analysis of cytokines and NO₂⁻/NO₃⁻: The following cytokines were measured using commercially available ELISA kits (R&D Systems, Minneapolis, Minn.): TNF, IL-10, and IL-6. Nitric oxide was measured as NO₂⁻/NO₃⁻ by the nitrate reductase method using a commercially available kit (Cayman Chemical, Ann Arbor, Mich.). Aspartate aminotransferase (AST) was measured using a commercially available kit according to manufacturer's instructions.

Mathematical model of acute inflammation.

We constructed a mathematical model of acute inflammation that incorporates key cellular and molecular components of the acute inflammatory response. In this model, pathogen-derived products, trauma, and hemorrhage are initiators of inflammation. (We note that hemorrhage is always accompanied by trauma). The mathematical model consists of a system of 17 ordinary differential equations that describe the time course of key components of the acute inflammatory response in terms of concentrations. Included in these equations are two systemic variables that represent mean arterial blood pressure and global tissue dysfumction and damage.

The differential equations were solved numerically using the software and freeware well within the skill of the art. Each equation was constructed from known interactions among model components as documented in the existing scientific literature. In deriving the mathematical model, we balanced biological realism with simplicity. Our goal was to find a fixed set of parameters that would qualitatively reproduce many known scenarios of inflammation found in the literature, correctly describe our data, and be able to make novel predictions to be tested experimentally, following the above guidelines and fitting the above inventive disclosure into appropriate software tools.

The model and parameters were specified in three stages. In the preliminary stage, the model was constructed so it could reproduce qualitatively several different scenarios that exist in the literature. In this stage, direct values of parameters such as cytokine half-lives were used when available. The resulting qualitatively correct model was then calibrated to experimental data from the three different inflammatory paradigms described above. In the second stage, the model was matched to our experimental data by adjusting the parameters using our knowledge of the biological mechanisms together with the dynamics of the model, to attain desired time course shapes. In the third stage, the parameters were optimized using a stochastic gradient descent algorithm that was implemented in appropriate software. The automated optimization procedure involved optimal adjustments of the scales of each of the analytes. The model was trained on data sets for four separate scenarios and then used to predict a fifth scenario. The statistical analysis of the model's ability to account for the data was performed with the S-Plus statistical and programming package (Statistical Sciences, Inc., Seattle, Wash.).

Results

We considered three distinct inflammatory paradigms: endotoxemia, surgical trauma, and surgical trauma followed by hemorrhagic shock. Four analytes—TNF-α, IL-10, IL-6, and a stable reaction product of NO—NO₂⁻/NO₃⁻—were measured in all scenarios. These four analytes were chosen because they represent a diverse selection of the main responders of the early inflammatory response, and are produced in a rapid (TNF, IL-10), intermediate (IL-6), and slow (NO₂⁻/NO₃⁻) time scale. As we will show, even with this limited data set, the relevant biological mechanisms and the mathematical model are severely constrained.

Kinetics of Cytokine and NO₂⁻/NO₃⁻ Production in Mouse Endotoxemia

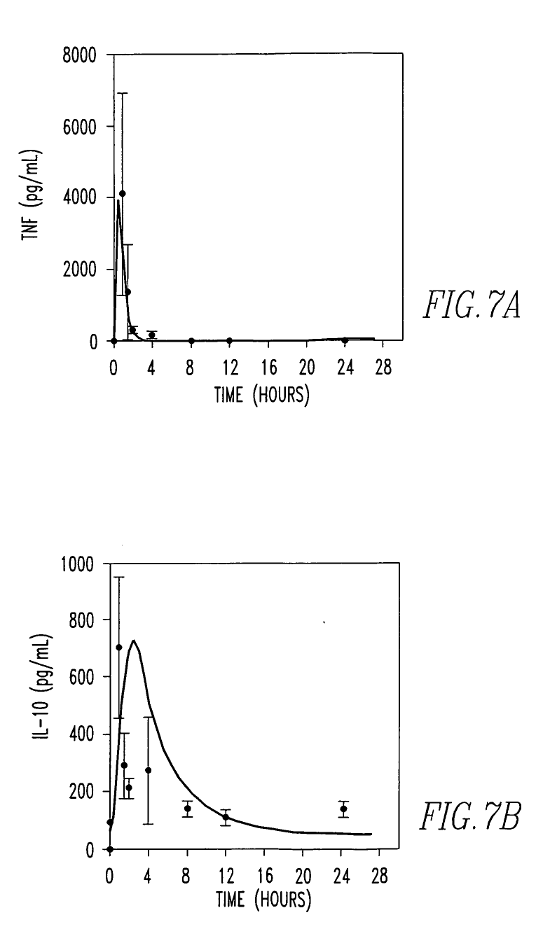

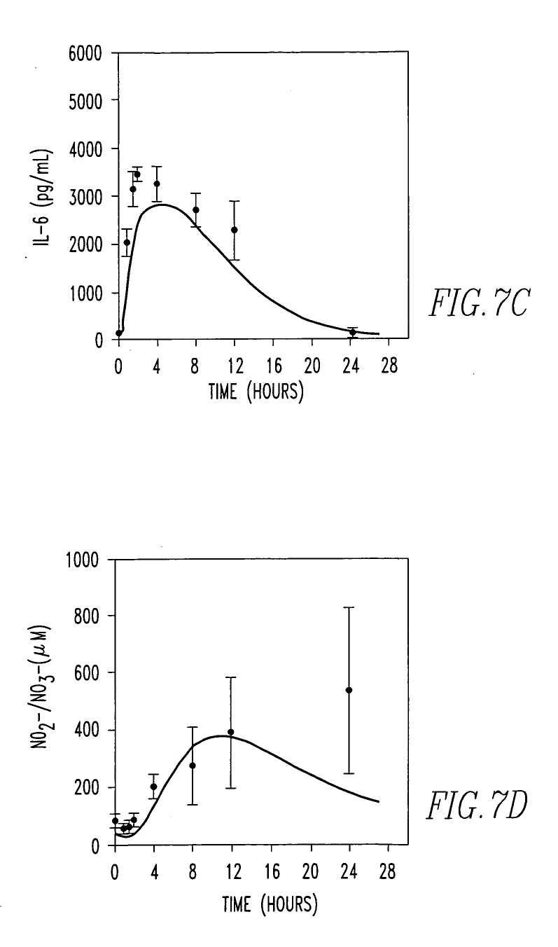

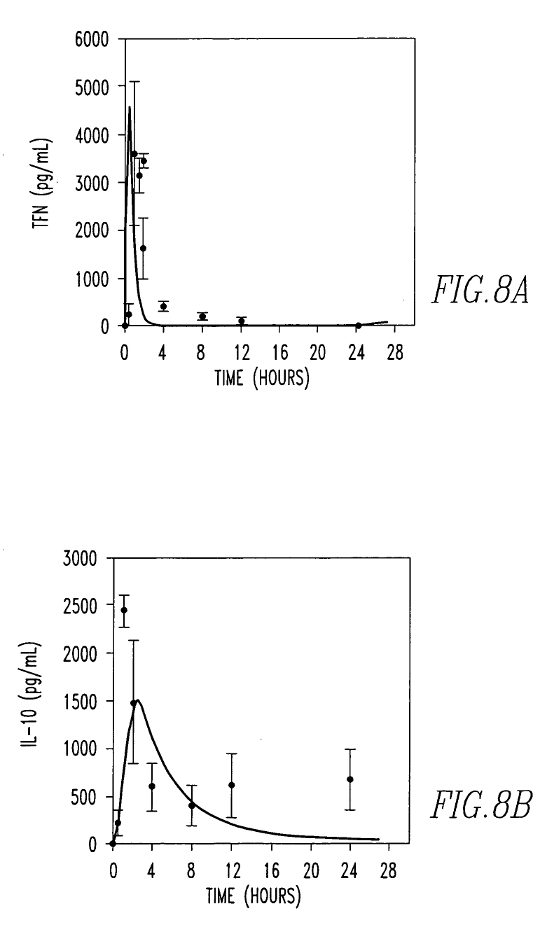

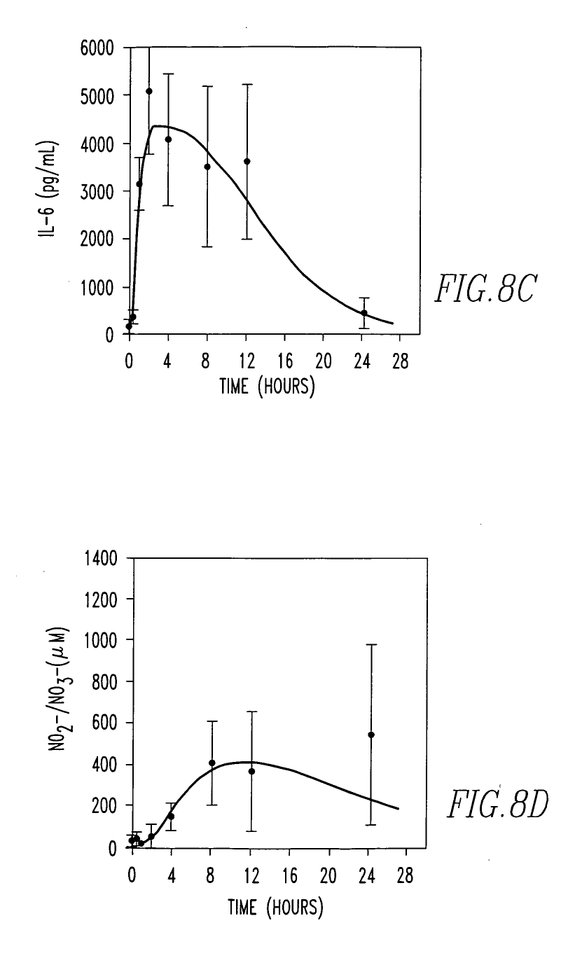

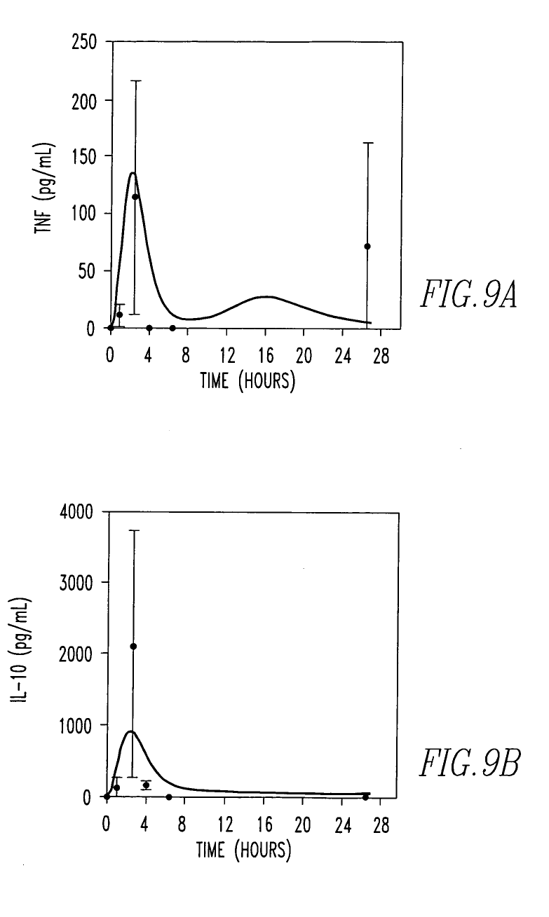

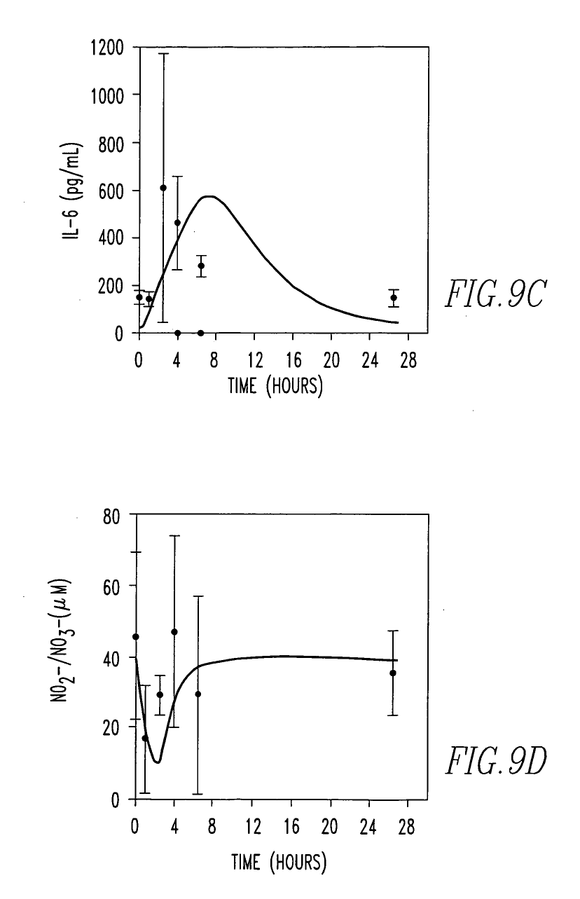

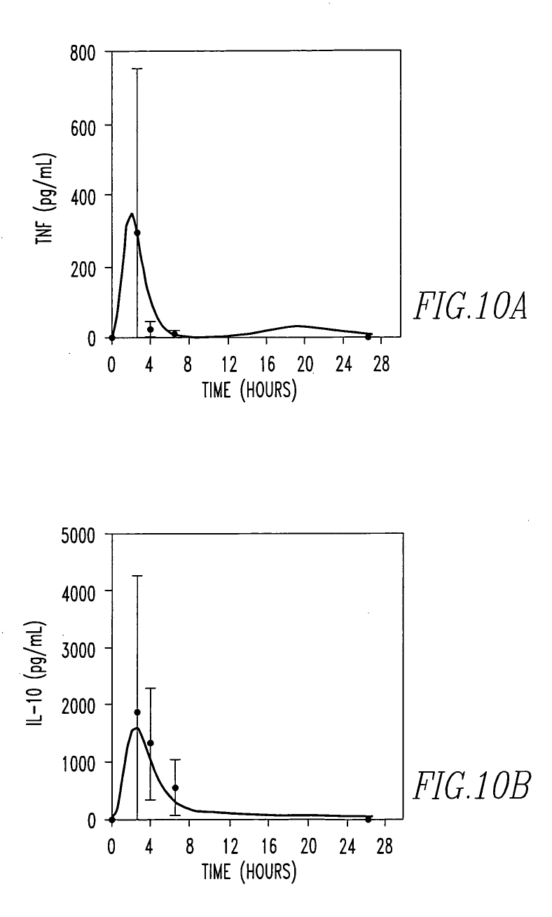

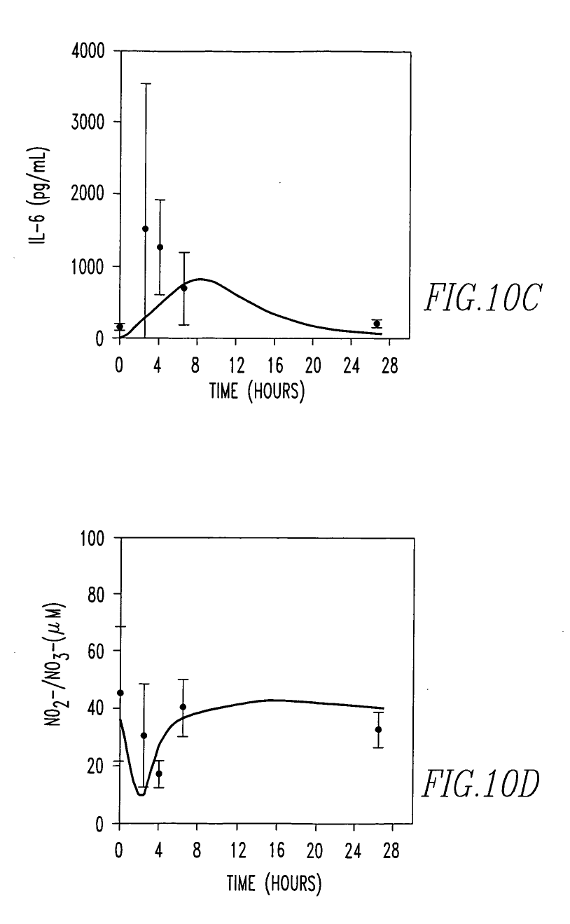

Endotoxemia, in which LPS is directly introduced into an animal, is a highly reproducible means for inducing acute systemic inflammation. FIG. 7 shows the experimental data (filled circles) from C57B1/6 mice given a sub-lethal (3 mg/kg) dose of LPS. FIG. 8 shows the results for a dose of 6 mg/kg LPS. Circulating levels of TNF and IL-10 increase rapidly and decay quickly, whereas IL-6 levels peak at approximately 2-3 h and decay more slowly. The levels of NO, measured as the stable reaction product NO₂⁻NO₃⁻, remain elevated for 24 h. Levels of TNF and NO₂⁻/NO₃⁻ seem to be saturated at 3 mg/kg whereas IL-6 and IL-10 saturate at 6mg/kg (FIG. 8): at 12 mg/kg (FIG. 9), the levels of most analytes are not much higher as compared to those of animals treated with 6 mg/kg LPS.

Kinetics of Cytokine and NO Production in Mouse Trauma/Hemorrhage

Trauma and hemorrhagic shock cause many of the same qualitative inflammatory consequences as endotoxemia, though with different kinetics and magnitude. Clinically, hemorrhagic shock often occurs in association with tissue trauma. We examined the inflammatory response to surgery alone and to surgery followed by hemorrhage and resuscitation. Normal, non-manipulated mice had low levels of cytokines in their serum (data not shown). Surgical trauma alone resulted in elevated circulating levels of the measured cytokines (FIG. 10). In contrast to endotoxemia, NO₂⁻/NO₃⁻ levels following trauma first decrease and then rise. We also note that there is a delay of approximately two hours before the cytokines respond. The absolute and relative peak levels differ significantly from endotoxemia. Compared to endotoxemia at 3 mg/kg, TNF peak level in trauma is approximately 20 to 40 times lower, IL-6 is approximately 7 times lower and IL-10 levels are slightly higher. TNF also has a secondary peak at 24 hours in trauma.

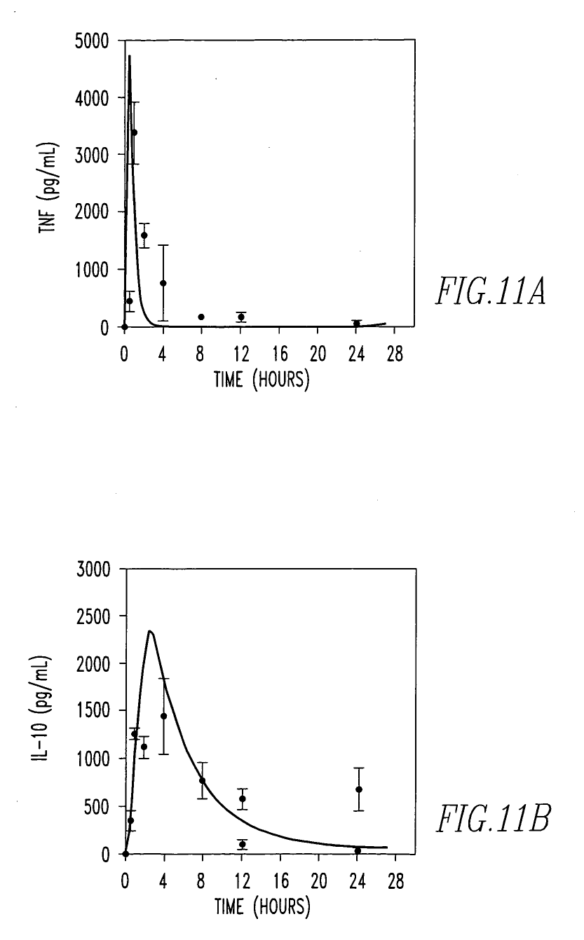

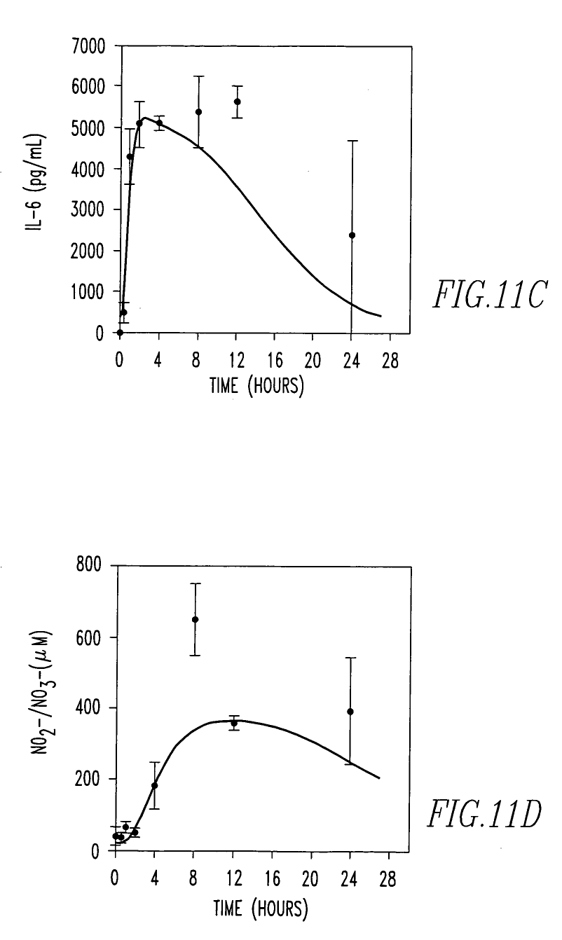

We also examined the effect of combined surgery and hemorrhage (FIG. 11). Animals subjected to this double insult had higher peak levels of TNF and IL-6, but similar or slightly higher levels of IL-10 as compared to trauma alone. NO₂⁻/NO₃⁻ has approximately the same form. However, we note that the experimental spread in the data is very large near the peaks. Although the data exhibit large variability at these points, the timing of these events is quite precise, possibly indicating that timing rather than amplitude may be a more salient marker for these diverse shock states.

Generation of a Mathematical Model of Acute Inflammation

The dynamics of the measured analytes for these three experimental paradigms exhibit significant differences, though they also share qualitative similarity. We propose that the observed differences in the inflammatory responses are due only to differences in the initiating insult: pathogen derived products vs. tissue trauma and/or blood loss. We further propose that once set in motion, the inflammatory response will follow a path determined by universal physiological mechanisms.

To support our hypotheses, we constructed a mathematical model that incorporates known physiological interactions between the various elements of the immune system. In the model, neutrophils and macrophages are activated directly by bacterial endotoxin (lipopolysaccharide [LPS]) or indirectly by various stimuli elicited systemically upon trauma and hemorrhage. Although not included explicitly in our model, early effects such as mast cell degranulation and complement activation are incorporated implicitly in the dynamics of our endotoxin and cytokine variables. These stimuli, including endotoxin, enter the systemic circulation quickly and activate circulating monocytes and neutrophils. Activated neutrophils also reach compromised tissue by migrating along a chemoattractant gradient.

Once activated, macrophages and neutrophils produce and secrete effectors that activate these same cells and also other cells, such as endothelial cells. Pro-inflammatory cytokines—TNF, IL-6, and IL-12 in our mathematical model—promote immune cell activation and pro-inflammatory cytokine production. The concurrent production of anti-inflammatory cytokines counterbalances the actions of pro-inflammatory cytokines. In an ideal situation, these anti-inflammatory agents serve to restore homeostasis. However, when overproduced, they may lead to detrimental immunosuppression.

Our model includes a fast-acting anti-inflammatory cytokine, IL-10, and a slower-acting anti-inflammatory activity encompassing active TGF-β, soluble receptors for pro-inflammatory cytokines, and cortisol. We note that while activated TGF-β only has a lifetime of a few minutes, latent TGF-P is ubiquitous and can be activated either directly or indirectly by other slower agents such as IL-6 or NO.

Pro-inflammatory cytokines also induce macrophages and neutrophils to produce free radicals. In our model, inducible NO synthase (iNOS)-derived NO is directly toxic to bacteria and indirectly to host tissue. Although the actions of superoxide (O₂⁻) and other lytic mechanisms do not appear explicitly in the model, their activity is accounted for implicitly through the pro-inflammatory agents. In the model, the actions of these products that can cause direct tissue dysfunction or damage are subsumed by the action of each cytokine directly. The induced damage can incite more inflammation by activating macrophages and neutrophils. However, NO can also protect tissue from damage induced by shock, even though overproduction of this free radical causes hypotension. Pro-inflammatory cytokines also reduce the expression of endothelial nitric oxide synthase (eNOS), thereby increasing tissue dysfunction.

The response to trauma (FIG. 10) exhibits a different time course from endotoxemia (FIGS. 7, 8, and 9). In endotoxemia, the model assumes that LPS enters the bloodstream and incites a system-wide response. Lipopolysaccharide is cleared in approximately one hour. Circulating neutrophils are activated directly and produce TNF and IL-10. The newly produced TNF combines with LPS to activate macrophages that then secrete TNF, IL-6, IL-12 and IL-10. Activated neutrophils, macrophages, and endothelial cells produce NO through iNOS. The model assumes that locally produced NO is eventually detected as the measured serum end products NO2−/NO3−, and this process depends on the differential induction of iNOS in various organs over time. In order for TNF to rise and fall within a few hours as it does in FIG. 7, the model required an inhibitory agent to suppress TNF production; this was accounted for by IL-10 and other slow anti-inflammatory cytokines including IL-6. Previous work has indicated that IL-6 may exert both pro- and anti-inflammatory properties. We believe this anti-inflammatory action could be mediated by inducing or activating TGF-β on the surface of neutrophils and macrophages, as has been shown for cytokines such as interferon. To account for the saturation of IL-6 for LPS levels beyond 6 mg/kg, in the model, we suggest that IL-6 also can act as an anti-inflammatory cytokine and inhibit production of itself. IL-10 is inhibited by IL-12 and stimulated by TGF-p that can come from various sources.

The response to trauma (FIG. 9) exhibits a different time course from endotoxemia (FIGS. 7, 8, and 11). To account for these differences in the model, we assume that localized trauma first induces platelets to release TGF-β which then chemoattracts circulating neutrophils to the site of injury. Simultaneously, elements associated with trauma and dysfunctional and/or damaged tissue (possibly HMG-B1) are released and activate the neutrophils when they arrive. The trauma-induced products combine with TNF to activate local macrophages to produce IL-6 and IL-10. In order to achieve the massive release of IL-10 in comparison to IL-6 and TNF in the model, we assumed that the released TGF-β induces activated macrophages to produce IL-10. We also assume that trauma causes a severe drop in eNOS (or eNOS-derived NO, e.g. by the rapid reduction in availability of L-arginine) to account for the dip in NO₂⁻/NO₃⁻; it is known that trauma patients exhibit reduced systemic NO2−/NO3− as compared to uninjured controls.

The model assumes that blood loss in hemorrhage causes some tissue damage as well as directly contributing to neutrophil and macrophage activation. This causes a greater release of TNF, which in turn induces higher IL-10 and IL-6 release. The model predicts that an increase in TNF and IL-6 will be accompanied by an increase in IL-10, though the spread in the data is too large to corroborate this prediction.

The following paragraphs identify additional aspects of the in silico design of clinical trials.

We introduce and evaluate the concept of conducting a randomized clinical trial in silico based on simulated patients generated from a mechanistic mathematical model of bacterial infection, the acute inflammatory response, global tissue dysfunction, and a therapeutic intervention. Trial populations are constructed to reflect heterogeneity in bacterial load and virulence, as well as propensity to mount and modulate an inflammatory response. We constructed a cohort of 1,000 trial patients submitted to therapy with one of three different doses of a neutralizing antibody directed against tumor necrosis factor (anti-TNF), for 6, 24, or 48 hours. We present cytokine profiles over time and expected outcome for each cohort. We identify subgroups with high propensity for being helped or harmed by the proposed intervention, and identify early serum markers for each of those subgroups.

The mathematical simulation confirms the inability of simple markers to predict outcome of sepsis. The simulation separates clearly cases with favorable and unfavorable outcome on the basis of global tissue dysfunction. Control survival was 62.9% at 1 week. Depending on dose and duration of treatment, survival ranged from 57.1% to 80.8%. Higher doses of anti-TNF, although effective, also result in considerable harm to patients. A statistical analysis based on a simulated cohort identified markers of favorable or adverse response to anti-TNF treatment.

A mathematical simulation of anti-TNF therapy identified clear windows of opportunity for this intervention, as well as populations that can be harmed by anti-TNF therapy. The construction of in silico clinical trial could provide profound insight into the design of clinical trials of immunomodulatory therapies, ranging from optimal patient selection to individualized dosage and duration of proposed therapeutic interventions.

The management of conditions associated with an intense inflammatory response such as severe trauma and sepsis represents a major challenge in the care of the critically ill. There is an emerging consensus that the acute inflammatory response to major stress might be inappropriate or lead to undesirable outcomes in patients initially resuscitated successfully. In the last two decades, much has been learned regarding cellular and molecular mechanisms of the acute inflammatory response. This progress has led to considerable efforts and resources to develop interventions that modulate the acute inflammatory response and positively impact outcome in these patients. Except for recombinant human activated protein C (drotrecogin alfa [activated]) and low-dose steroids, this knowledge has not led to effective immunomodulatory therapies; consequently, a significant effort to address the issue of target confirmation and trial design has ensued. This situation is especially vexing considering that a reasonable therapeutic rationale was supported by animal and early phase human studies for dozens of interventions that failed when evaluated in phase III.

Several researchers have proposed a variety of reasons to explain the incongruence between results and expectations. We propose that a key reason for this conundrum is the difficulty to predict the impact of modifying single components of the highly complex, non-linear, and redundant inflammatory response. The consequences of failing to take a systems-oriented approach to understanding and predicting the time-course of complex diseases are various and significant. Indeed, prediction of the behavior of such systems derived from localized insights gathered from limited experiments or observations pertaining to individual components on such systems may be impossible, however accurate these isolated observations may be. Meteorologists, engineers, physicists, and other scientists examining complex systems make extensive use of models, simplified representations of those complex systems, to shed useful insight on the behavior of such systems.

We sought to adopt a similar approach and conduct a practical demonstration of modeling a clinical trial in silico, by examining a therapy that had initial great promise in the setting of animal models of sepsis, but failed in large, randomized clinical trials to meet generally accepted criteria for efficacy. Accordingly, we focused on the consequences of the administration to sepsis patients of a neutralizing antibody directed against the pro-inflammatory cytokine tumor necrosis factor (anti-TNF). After promising non-human primate results, pooled outcome of no fewer than 11 clinical trials in 7,265 patients showed a consistent absolute reduction in mortality of approximately 3.2% (p=0.006) favoring treatment with anti-TNF antibodies, a disappointing result in light of the effect expected from pre-clinical studies. Efforts to select populations that would demonstrate a convincing benefit from anti-TNF have not met expectations either.

We wish to illustrate insights that mathematical models could provide in elucidating the reasons for the disappointing results of this particular agent and, more generally, in the design of fuiture trials, especially regarding drug dosing, duration of therapy, and interaction among co-interventions.

We initially designed a mechanistic model of the acute inflammatory response based on information available from the existing literature on the roles of key cellular and molecular effectors in response to a bacterial pathogen. We constructed a population of virtual patients differing in their initial bacterial load, bacterial virulence, time of initiation of intervention, and genetic ability to generate effectors in response to stress. We compared outcomes across several treatment arms and identified determinants of favorable and unfavorable outcomes.

Because the acute inflammatory response is comprised of a large number of components that each have specific roles, yet are highly interactive, we chose to model this dynamical system with a system of differential equations, one for each component that we chose to simulate. Each equation describes the level or concentration of components over time resulting from their interaction with other components following the principle of mass-action. We chose to represent the system at this level because serum levels of cytokines, for example, are well known to correlate with outcome in septic patients, clinical measurements are usually obtained from blood, and chemotherapeutic interventions are typically administered intravenously. Limitations resulting from this choice are discussed below. The strengths of such an approach are several, in that it 1) provides an intuitive means to translate mechanistic concepts into a mathematical framework, 2) can be analyzed using a large body of existing techniques, 3) can be numerically simulated easily and inexpensively on a desktop computer, 4) provides both qualitative and quantitative predictions, and 5) allows expansion to higher levels of complexity.

Initial values for rate constants were determined empirically so that the model would qualitatively reproduce observed literature data in mice administered endotoxin or subjected to cecal ligation and puncture. Some rate constants, such as cytokine half-lives, were directly extracted from the literature.

We generated a study population of 1,000 virtual patients. Pathogen characteristics (growth rate and initial load) were chosen to result in a survival of approximately 60%. We varied the delay before medical consultation, and thus eligibility for treatment, reasoning that the distribution of the delays to medical consultation after onset of infection was related to initial pathogen load and virulence (i.e. sicker cases would generally consult earlier). To simulate genetic diversity of the study population we randomly varied individual propensity of immune cells to generate effector molecules (pro-inflammatory such as TNF and Interleukin [IL]-6), anti-inflammatory, and nitric oxide synthase activity) from ±25% of baseline as dictated by literature data. Those variations were sufficient to explain wide swings in individual serum levels of effectors.

We wished to illustrate the application of mathematical modeling to optimizing the design of a clinical trial. We achieved this demonstration in two steps. First, we identified administration strategies that would result in the best outcomes for the entire cohort. Second, we illustrate how the simulation can help with patient selection, given a treatment administration regimen. Importantly, our goal was specifically not the optimization of treatment regimen to individuals, although this constitutes another potential application of our simulation.

To identify optimal dosing and duration of administration strategies, we submitted the virtual cohort of 1,000 patients to nine interventions with anti-TNF. We varied the duration of administration of anti-TNF (6h, 24 h, or 48 h). Comparatively, the half-life of anti-TNF antibodies in naive patients is 40 to 50 hours. We simulated the binding of serum TNF with three different “doses” of anti-TNF (2, 10, and 20 arbitrary units). Depending on dose, TNF neutralization varied from 18.6% to 55.5% of total TNF produced in controls. A clear correlation with published reports is difficult as these do not typically report areas under the curve, and do not always distinguish between biologically active TNF, TNF bound by antibody and TNF bound by specific soluble receptors. Death was determined by the inability of the individuals to clear more than 50% of maximal sustained tissue dysfunction at one week. Such a definition segregated the population into two outcome groups.

Trial optimization involves selecting a dosing strategy that optimizes outcome in a cohort of patients, and then selecting patients that would benefit from treatment while avoiding treating patients for which treatment would have either no effect or cause harm. The optimal treatment administration scheme has already been determined as part of prior results (see section above). To select patients that would most benefit from this treatment, we constructed a multinomial logistic model with a four-valued outcome variable: 1) helped by treatment (survives but would have died without treatment), 2) survives irrespective of treatment, 3) dies irrespective of treatment, and 4) is harmed (dies because of treatment). Independent variables were chosen at the time of disease detection (the earliest possible treatment opportunity) and 60 minutes later, reflecting the possibility of using short-term trends in analytes and assuming rapid diagnostic capabilities. Variables included serum TNF, anti-inflammatory activity, long-acting pro-inflammatory cytokine (IL-6), their ratios and products, activated protein C, thrombin, as well as blood pressure and cell counts of activated neutrophils. The statistical model was validated in a different population of 1,000 simulated cases. All predictions from the statistical model relate to the validation population.

We wrote our own software for the simulations and analyses (JB, RK, GC). Statistical analyses and multivariate statistical models were conducted in SPSS, (SPSS, Inc, Chicago, Ill.).

The results of the above-described simulated clinical trial have been omitted here, inasmuch as the intention is to disclose the approach to the simulated clinical trial, not necessarily the results per se. However, the inventors do represent herewith that data were determined which are scheduled for publication in due course.

All the above disclosure throughout this specification should be understood to extend to both chronic and acute inflammation, and to extensions of the life/death paradigm which foresees morbidity versus wellness.

Although the invention has been described with particularity above, in reference to specific methods, materials, and examples, the invention is only to be limited insofar as is set forth in the accompanying claims.

1. A method for prognosing the life or death outcome of an animal or patient in which bacterial infection or inflammation is present, comprising measuring at least two physiological factors significant to the progress of bacterial infection or inflammation and predicting the likelihood of death.

2. The method according to claim 1, wherein the likelihood of death is governed by a damage function dD/dt, and wherein the damage function dD/dt is determined according to the differential equations:
$$\begin{matrix}\frac{ⅅ P}{ⅅ t} = {k}_{p} P ( 1 - {k}_{Ps} P ) - ( {k}_{PM} {M}_{a} + {k}_{PO2} {O}_{2} + {k}_{PNO} NO + & ( {1}^{′} ) \\ AB ( t ) ) P + {S}_{P} ( t ) & \\ \frac{ⅅ PE}{ⅅ t} = ( {k}_{P} {M}_{a} + {k}_{PO2} {O}_{2} + {k}_{PNO} NO + AB ( t ) P - {k}_{PE} PE + {S}_{PE} ( t ) & ( {2}^{′} ) \\ \frac{ⅅ {M}_{r}}{ⅅ t} = - ( {k}_{MP} p + {k}_{MPE} PE + {k}_{MD} D ) ( f ( {C}_{p} ) + f ( IL - 6 ) ) {f}_{s} ( {C}_{a} ) + & ( {3}^{′} ) \\ {k}_{Mg} f ( {M}_{a} + {C}_{p} + NO + PE ) - {k}_{M} {M}_{r} & \\ \frac{ⅅ {M}_{a}}{ⅅ t} = ( {k}_{m p} p + {k}_{pe} PE + {k}_{md} D ) ( f ( {C}_{p} ) + f ( IL - 6 ) ) {f}_{s} ( {C}_{a} ) - {k}_{ma} {M}_{a} & ( {4}^{′} ) \\ \frac{ⅅ N}{ⅅ t} = ( {k}_{NP} P + {k}_{NPE} PE + {k}_{NCP} {C}_{P} + {k}_{NIL - 6} IL - 6 + {k}_{ND} D ) N - & ( {5}^{′} ) \\ ( {k}_{NNO} NO + {k}_{NO2} O2 ) N - {k}_{N} {f}_{s} ( {C}_{p} ) N & \\ \frac{ⅅ {O}_{2}}{ⅅ t} = ( ( {k}_{O2N} N + {k}_{O2M} {M}_{a} ) ( f ( {C}_{p} ) + f ( IL - 6 ) ) + & ( {6}^{′} ) \\ {k}_{O2NP} NP ) {f}_{s} ( {C}_{a} ) - {k}_{O2} {O}_{2} & \\ \frac{ⅅ N}{\frac{O}{ⅅ t}} = ( {k}_{NON} N + {k}_{NOM} {M}_{a} ) ( f ( {C}_{p} ) + f ( IL - 6 ) ) {f}_{s} ( {C}_{a} ) - {k}_{NO} NO & ( {7}^{′} ) \\ \frac{ⅅ {C}_{p}}{ⅅ t} = ( {k}_{CpN} N + {k}_{CpM} {M}_{a} ) ( 1 + {k}_{CPn} f ( {C}_{p} ) ) {f}_{s} ( {C}_{a} ) - {k}_{Cp} {C}_{p} & ( {8}^{′} ) \\ \frac{ⅅ I {L}_{-}}{\frac{6}{ⅅ t}} = {k}_{IL - 6 M} {M}_{a} {f}_{s} ( {C}_{a} ) - {k}_{IL - 6} IL - 6 & ( {9}^{′} ) \\ \frac{ⅅ {C}_{ar}}{ⅅ t} = ( {k}_{CaN} N + {k}_{CaM} {M}_{a} ) f ( {C}_{p} + NO + {O}_{2} ) - {k}_{Car} {C}_{ar} & ( {10}^{′} ) \\ \frac{ⅅ {C}_{a}}{ⅅ t} = {C}_{ar} - {k}_{Ca} {C}_{a} + {S}_{PC} ( t ) & ( {11}^{′} ) \\ \frac{ⅅ TF}{ⅅ t} = ( {k}_{TFPE} PE + {k}_{TFCp} {C}_{p} + {k}_{TFIL - 6} IL - 6 ) {f}_{s} ( PC ) - & ( {12}^{′} ) \\ {k}_{TF} TF - ktf ( t ) TF & \\ \frac{ⅅ TH}{ⅅ t} = TF ( 1 + {k}_{THn} TH ) - {k}_{TH} TF & ( {13}^{′} ) \\ \frac{ⅆ TH}{ⅆ T} = TF ( 1 + {k}_{THn} TH ) - {k}_{TH} TF & \\ \frac{ⅅ PC}{ⅅ t} = {k}_{PCTH} TH - {k}_{PC} PC + {S}_{PC} ( t ) & ( {14}^{′} ) \\ \frac{ⅅ BP}{ⅅ t} = {k}_{BP} ( 1 - BP ) - {k}_{BPO2} {O}_{2} {f}_{s} ( NO ) + {k}_{BPCp} {C}_{p} + {k}_{BPTH} TH ) BP & ( {15}^{′} ) \\ \frac{ⅅ D}{ⅅ t} = {k}_{DBP} ( 1 - BP ) + {k}_{DCp} {C}_{p} + {k}_{DO2} {O}_{2} + {k}_{DNO} NO / ( 1 + NO ) + & ( {16}^{′} ) \\ {k}_{DEqg} ( {O}_{2} , NO ) - {k}_{D} D & \end{matrix}$$

3. The method according to claim 2, wherein the damage function is evidenced by a value selected from the group consisting of the ratio of IL-6/NO and the ratio of IL-6/IL-10 at a predetermined point after the onset of infection.

4. The method according to claim 3, wherein the damage function is evidenced according to the ratio of IL-6/NO and further wherein when the IL-6/NO ratio is <8 at 12 hours post infection, the likelihood of mortality is about 60%.

5. The method according to claim 3, wherein the damage function is evidenced according to the ratio of IL-6/NO and further wherein when the IL-6/NO ratio is <4 at 24 hours post infection, the likelihood of mortality is about 52%.

6. The method according to claim 3, wherein the damage function is evidenced according to the ratio of IL-6/IL- 10 and further wherein when the IL-6/IL-10 ratio is <7.5 at 24 hours post infection, the likelihood of mortality is about 68%.

7. A method for evaluating a drug candidate, comprising enhancing the meaning of an animal model study by comparing inflammation or infection data from said animal study with human data collected from human clinical trials, said human data being considered according to the equations:
$$\begin{matrix}\frac{ⅅ P}{ⅅ t} = {k}_{p} P ( 1 - {k}_{Ps} P ) - ( {k}_{PM} {M}_{a} + {k}_{PO2} {O}_{2} + {k}_{PNO} NO + & ( {1}^{′} ) \\ AB ( t ) ) P + {S}_{P} ( t ) & \\ \frac{ⅅ PE}{ⅅ t} = ( {k}_{P} {M}_{a} + {k}_{PO2} {O}_{2} + {k}_{PNO} NO + AB ( t ) P - {k}_{PE} PE + {S}_{PE} ( t ) & ( {2}^{′} ) \\ \frac{ⅅ {M}_{r}}{ⅅ t} = - ( {k}_{MP} p + {k}_{MPE} PE + {k}_{MD} D ) ( f ( {C}_{p} ) + f ( IL - 6 ) ) {f}_{s} ( {C}_{a} ) + & ( {3}^{′} ) \\ {k}_{Mg} f ( {M}_{a} + {C}_{p} + NO + PE ) - {k}_{M} {M}_{r} & \\ \frac{ⅅ {M}_{a}}{ⅅ t} = ( {k}_{m p} p + {k}_{pe} PE + {k}_{md} D ) ( f ( {C}_{p} ) + f ( IL - 6 ) ) {f}_{s} ( {C}_{a} ) - {k}_{ma} {M}_{a} & ( {4}^{′} ) \\ \frac{ⅅ N}{ⅅ t} = ( {k}_{NP} P + {k}_{NPE} PE + {k}_{NCP} {C}_{P} + {k}_{NIL - 6} IL - 6 + {k}_{ND} D ) N - & ( {5}^{′} ) \\ ( {k}_{NNO} NO + {k}_{NO2} O2 ) N - {k}_{N} {f}_{s} ( {C}_{p} ) N & \\ \frac{ⅅ {O}_{2}}{ⅅ t} = ( ( {k}_{O2N} N + {k}_{O2M} {M}_{a} ) ( f ( {C}_{p} ) + f ( IL - 6 ) ) + & ( {6}^{′} ) \\ {k}_{O2NP} NP ) {f}_{s} ( {C}_{a} ) - {k}_{O2} {O}_{2} & \\ \frac{ⅅ N}{\frac{O}{ⅅ t}} = ( {k}_{NON} N + {k}_{NOM} {M}_{a} ) ( f ( {C}_{p} ) + f ( IL - 6 ) ) {f}_{s} ( {C}_{a} ) - {k}_{NO} NO & ( {7}^{′} ) \\ \frac{ⅅ {C}_{p}}{ⅅ t} = ( {k}_{CpN} N + {k}_{CpM} {M}_{a} ) ( 1 + {k}_{CPn} f ( {C}_{p} ) ) {f}_{s} ( {C}_{a} ) - {k}_{Cp} {C}_{p} & ( {8}^{′} ) \\ \frac{ⅅ I {L}_{-}}{\frac{6}{ⅅ t}} = {k}_{IL - 6 M} {M}_{a} {f}_{s} ( {C}_{a} ) - {k}_{IL - 6} IL - 6 & ( {9}^{′} ) \\ \frac{ⅅ {C}_{ar}}{ⅅ t} = ( {k}_{CaN} N + {k}_{CaM} {M}_{a} ) f ( {C}_{p} + NO + {O}_{2} ) - {k}_{Car} {C}_{ar} & ( {10}^{′} ) \\ \frac{ⅅ {C}_{a}}{ⅅ t} = {C}_{ar} - {k}_{Ca} {C}_{a} + {S}_{PC} ( t ) & ( {11}^{′} ) \\ \frac{ⅅ TF}{ⅅ t} = ( {k}_{TFPE} PE + {k}_{TFCp} {C}_{p} + {k}_{TFIL - 6} IL - 6 ) {f}_{s} ( PC ) - & ( {12}^{′} ) \\ {k}_{TF} TF - ktf ( t ) TF & \\ \frac{ⅅ TH}{ⅅ t} = TF ( 1 + {k}_{THn} TH ) - {k}_{TH} TF & ( {13}^{′} ) \\ \frac{ⅆ TH}{ⅆ T} = TF ( 1 + {k}_{THn} TH ) - {k}_{TH} TF & \\ \frac{ⅅ PC}{ⅅ t} = {k}_{PCTH} TH - {k}_{PC} PC + {S}_{PC} ( t ) & ( {14}^{′} ) \\ \frac{ⅅ BP}{ⅅ t} = {k}_{BP} ( 1 - BP ) - {k}_{BPO2} {O}_{2} {f}_{s} ( NO ) + {k}_{BPCp} {C}_{p} + {k}_{BPTH} TH ) BP & ( {15}^{′} ) \\ \frac{ⅅ D}{ⅅ t} = {k}_{DBP} ( 1 - BP ) + {k}_{DCp} {C}_{p} + {k}_{DO2} {O}_{2} + {k}_{DNO} NO / ( 1 + NO ) + & ( {16}^{′} ) \\ {k}_{DEqg} ( {O}_{2} , NO ) - {k}_{D} D & \end{matrix}$$
so as to impute damage function calculations from the human data into the animal data and to enhance prediction of efficacy of said drug candidate.

8. The method according to claim 7 wherein a mathematical model describing the acute inflammatory cascade, and that culminates in global tissue damage/dysfunction (D), is used to predict the required mechanism of action of a drug to be used to improve outcome of sepsis or trauma, and which drug is subsequently vetted in screening assays in vivo not only to confirm the mathematical model but to enhance, if applicable, the model for the purposes of evaluating said drug.

9. The method according to claim 7 wherein a mathematical model describing the acute inflammatory cascade, and that culminates in global tissue damage/dysfunction (D), is used to predict the required mechanism of action of a drug to be used to improve outcome of sepsis or trauma, and which drug is subsequently vetted in screening assays both in vivo and in vitro not only to confirm the mathematical model but to enhance, if applicable, the model for the purposes of evaluating said drug.

10. A method for evaluating a drug candidate, comprising enhancing the meaning of an animal model study by comparing inflammation or infection data from said animal study with human data collected from human clinical trials, said human data being considered according to the equations
$$\begin{matrix}{M}_{R}^{′} = - [ ( {k}_{MLPS} \frac{{LPS ( t )}^{2}}{1 + {( LPS ( t ) / {x}_{MLPS} )}^{2}} + {k}_{MD} \frac{{D}^{4}}{{x}_{MD}^{4} + {D}^{4}} ) \times & {1}^{′} ’ \\ ( \frac{{TNF}^{2}}{{x}_{{MTNF}^{2}} + {TNF}^{2}} + {k}_{M6} \frac{{IL6}^{2}}{{x}_{M6}^{2} + {IL6}^{2}} ) + & \\ {k}_{MTR} TR ( t ) + {k}_{MB} {f}_{B} ( B ) ] \frac{1}{1 + {( IL10 / {x}_{M10} )}^{2}} {M}_{R} - & \\ {k}_{MR} ( {M}_{R} - {S}_{M} ) & \\ {M}_{A}^{′} = [ ( {k}_{MLPS} \frac{{LPS ( t )}^{2}}{1 + {( LPS ( t ) / {x}_{MLPS} )}^{2}} + {k}_{MD} \frac{{D}^{4}}{{x}_{MD}^{4} + {D}^{4}} ) \times & {2}^{′} ’ \\ ( \frac{{TNF}^{2}}{{x}_{{MTNF}^{2}} + {TNF}^{2}} + {k}_{M6} \frac{{IL6}^{2}}{{x}_{M6}^{2} + {IL6}^{2}} ) + {k}_{MTR} TR ( t ) + {k}_{MB} {f}_{B} ( B ) ] & \\ \frac{1}{1 + {( IL10 / {x}_{M10} )}^{2}} {M}_{R} - {k}_{MA} MA & \\ {N}_{R}^{′} = - [ ( {k}_{NLPS} \frac{LPS ( t )}{1 + LPS ( t ) / {x}_{NLPS}} + {k}_{NTNF} \frac{TNF}{1 + TNF / {x}_{NTNF}} + & {3}^{′} ’ \\ {k}_{N6} \frac{{IL6}^{2}}{1 + {( IL6 / {x}_{N6} )}^{2}} + {k}_{ND} \frac{{D}^{2}}{1 + {( D / {x}_{ND} )}^{2}} + {k}_{NB} {f}_{B} ( B ) + & \\ {k}_{NTR} TR ( t ) ) \times \frac{1}{1 + {( IL10 / {x}_{N10} )}^{2}} {N}_{R} - {k}_{NR} ( {N}_{R} - {S}_{N} ) & \\ {N}_{A}^{′} = [ ( {k}_{NLPS} \frac{LPS ( t )}{1 + {( LPS ( t ) / {x}_{NLPS} )}^{2}} + {k}_{NTNF} \frac{TNF}{1 + TNF / {x}_{NTNF}} + & {4}^{′} ’ \\ {k}_{N6} \frac{{IL6}^{2}}{1 + {( IL6 / {x}_{N6} )}^{2}} + {k}_{ND} \frac{{D}^{2}}{1 + {( D / {x}_{ND} )}^{2}} + {k}_{NB} {f}_{B} ( B ) + & \\ {k}_{NTR} TR ( t ) ) \times \frac{1}{1 + {( IL10 / {x}_{N10} )}^{2}} {N}_{R} - {k}_{N} {N}_{A} & \\ {iNOSd}^{ ′} = ( {k}_{INOSN} {N}_{A} + {k}_{INSOM} {M}_{A} + {k}_{INOSEC} & {5}^{′} ’ \\ ( \frac{{TNF}^{2}}{1 + {( TNF / {x}_{INOSTNF} )}^{2}} + {k}_{INOS6} \frac{{IL6}^{2}}{1 + {( IL6 / {x}_{INOS6} )}^{2}} ) ) \times & \\ \frac{1}{1 + {( IL10 / {x}_{INOS10} )}^{2}} \frac{1}{1 + {( NO / {x}_{iNOSNO} )}^{4}} - {k}_{INOSd} i NOSd & \\ {iNOS}^{ ′} = {k}_{iNOS} ( iNOSd - iNOS ) & {6}^{′} ’ \\ {eNOS}^{ ′} = {k}_{ENOSEC} \frac{1}{1 + TNF / {x}_{ENOSTNF}} \frac{1}{1 + LPS ( t ) / {x}_{ENOSLPS}} & {7}^{′} ’ \\ \frac{1 }{1 + {( TR ( t ) / {x}_{ENOSTR} )}^{4}} - {k}_{ENOS} eNOS & \\ {NO}_{3}^{′} = {k}_{NO3} ( NO - {NO}_{3} ) & {8}^{′} ’ \\ {TNF}^{′} = ( {k}_{TNFN} {N}_{A} + {k}_{TNFN} {M}_{A} ) \frac{1}{1 + {( ( IL10 + CA ) / {x}_{TNF10} )}^{2}} & {9}^{′} ’ \\ \frac{IL6}{1 + {( IL6 / {x}_{TNF6} )}^{3}} - {k}_{TNF} TNF & \\ {IL6}^{′} = ( {k}_{6 N} {N}_{A} + {M}_{A} ) ( {k}_{6 M} + {k}_{6 TNF} \frac{{TNF}^{2}}{{x}_{6 TNF}^{2} + {TNF}^{2}} + {k}_{6 NO} & {10}^{′} ’ \\ \frac{{NO}^{2}}{{x}_{6 NO}^{2} + {NO}^{2}} ) \frac{1}{1 + {( ( CA + IL10 ) / {x}_{610} )}^{2}} + {k}_{6} ( {S}_{6} - IL6 ) & \\ {IL12}^{′} = {k}_{12 M} {M}_{A} \frac{1}{1 + {( IL10 / {x}_{1210} )}^{2}} - {k}_{12} IL12 & {11}^{′} ’ \\ {CA}^{′} = {k}_{CATR} A ( t ) - {k}_{CA} CA & {12}^{′} ’ \\ {IL10}^{′} = ( {k}_{10 N} {N}_{A} + {M}_{A} ( 1 + {k}_{10 A} A ( t ) ) ( {k}_{10 MR} + {k}_{10 TNF} & {13}^{′} ’ \\ \frac{{TNF}^{4}}{{x}_{10 TNF}^{4} + {TNF}^{4}} + {k}_{106} \frac{{IL6}^{4}}{{x}_{106}^{4} + {IL6}^{4}} ) ( 1 - {k}_{10 R} ) & \\ \frac{1}{1 + {( IL12 / {x}_{1012} )}^{4}} + {k}_{10 R} ) - {k}_{10} ( IL10 - {S}_{10} ) & \end{matrix}$$
so as to impute damage function calculations from the human data into the animal data and to enhance prediction of efficacy of said drug candidate.

11. The method according to claim 10 wherein a mathematical model describing the acute inflammatory cascade, and that culminates in global tissue damage/dysfunction (D), is used to predict the required mechanism of action of a drug to be used to improve outcome of sepsis or trauma, and which drug is subsequently vetted in screening assays in vivo not only to confirm the mathematical model but to enhance, if applicable, the model for the purposes of evaluating said drug.

12. The method according to claim 10 wherein a mathematical model describing the acute inflammatory cascade, and that culminates in global tissue damage/dysfunction (D), is used to predict the required mechanism of action of a drug to be used to improve outcome of sepsis or trauma, and which drug is subsequently vetted in screening assays both in vivo and in vitro not only to confirm the mathematical model but to enhance, if applicable, the model for the purposes of evaluating said drug.
